(19) United States
(12) Patent Application Publication
(10) Pub. No.: US 2023/0399671 A1
(43) Pub. Date: Dec. 14, 2023

(54) BETA-1,4 GALACTOSYLATION OF PROTEINS

(71) Applicant: University College Dublin, National University of Ireland, Dublin (IE)

(72) Inventors: Ioscani JIMENEZ DEL VAL, Dublin (IE); Itzcóatl Arturo GOMEZ AQUINO, Dublin (IE)

(73) Assignee: University College Dublin, National University of Ireland, Dublin (IE)

(21) Appl. No.: 18/041,352

(22) PCT Filed: Aug. 10, 2021
(86) PCT No.: PCT/EP2021/072287

§ 371 Date: Feb. 10, 2023

(30) Foreign Application Priority Data
Aug. 11, 2020 (GB) 2012512.6

(51) Int. Cl. C12P 21/00; C12N 9/10; C07K 16/00

(52) U.S. Cl. CPC C12P 21/005; C12N 9/1051; C12Y 204/01038; C12Y 204/01122; C07K 16/00; C07K 2317/41; C07K 2317/14; C07K 2317/94; C07K 2317/732; C07K 2317/734

(57) ABSTRACT

The present invention relates to a cell, wherein the cell is modified to: reduce O-GalNAc galactosylation activity in the cell by reduction of functional COSMC molecular chaperone in the cell and/or by reduction of functional T-synthase in the cell; and overexpress β1,4-galactosyltransferase in the cell; and associated methods, kits and uses.

This invention relates to a cell, wherein the cell is modified to enhance β-1,4 galactosylation of polypeptide product produced by the cell, a method of modifying a cell, and methods of producing a polypeptide product.

β-1,4 galactosylation is the major source of therapeutic protein variability and arises from even subtle process deviations during manufacturing operations. Nutrient availability, metabolite accumulation, temperature, pH and other bioprocess conditions vary during the large-scale cell culture processes whereby therapeutic proteins are manufactured. Variations in bioprocess conditions are widely known to heavily influence glycosylation, in particular, β-1,4 galactosylation. Reducing product heterogeneity is key to ensuring the safety and therapeutic efficacy of therapeutic proteins and is, thus, a fundamental aim of biopharmaceutical manufacturing operations.

The positive impact of β-1,4 galactosylation on therapeutic protein efficacy has only been reported recently. Therefore, the technologies developed to date have aimed to modulate β-1,4 galactosylation in order to reduce product heterogeneity or enhance the anti-inflammatory activity and serum half-life of biopharmaceuticals via increased sialylation. There are three broad technology classes that have been developed to influence the β-1,4 galactosylation of recombinant glycoproteins:

1. Uridine/Manganese/Galactose (UMG) Feeding.

Metabolic regulation of therapeutic glycoprotein β-1,4 galactosylation has been achieved by feeding biosynthetic precursors of UDP-Gal (the co-substrate required for galactosylation).

Within this strategy, feeding of uridine and galactose to cell culture enhances the intracellular availability of UDP-Gal, while manganese is added to enhance the activity of β4GalT1 (Mn2+ is the enzyme's catalytic co-factor). UMG feeding is performed throughout cell culture to achieve target β-1,4 galactosylation profiles on the recombinant protein. One study on UMG feeding achieved an increase in mAb β-1,4 galactosylation of 5.3% to 39% (6.8% bi-galactosylated glycans) for one cell line and 9% to 48% (9.7% bi-galactosylated glycans) for another cell line [1]. A second study achieved increases of 56% to 79% (24% bi-galactosylated glycans) for one cell line and 36% to 67% (19% bi-galactosylated glycans) for another [2].

However, there are three fundamental drawbacks associated with the UMG feeding strategy used to modulate β-1,4 galactosylation: (1) Only modest changes in β-1,4 galactosylation can be achieved with UMG feeding, in particular the gains in bi-galactosylated glycans; (2) UMG feeding has been reported to negatively impact the growth and therapeutic protein productivity of CHO cell culture. All three components involved in UMG feeding (uridine, manganese and galactose) are reported to reduce the growth rate of CHO cells and, in consequence, therapeutic protein yield; and (3) Extensive experimentation is required to identify optimal UMG feeding strategies that achieve the desired effects on β-1,4 galactosylation while minimising negative impacts on therapeutic protein yield. Despite the limitations of UMG feeding, it is used in industry because the strategy can be deployed to modulate the β-1,4 galactosylation of approved bioprocesses/therapeutic products.

2. Ectopic β4Ga1T Expression

Several studies have ectopically expressed human β4GalT1 in therapeutic protein producing CHO cell lines, mainly with the aim of increasing product sialylation. The extent of product β-1,4 galactosylation achieved through ectopic β4GalT expression ranges from 73%-98% and bi-galactosylation from 49%-87%, which is greater than that achieved through UMG feeding strategies. The work of Schulz et al. [25], in particular, achieves the highest gains in both overall product β-1,4 galactosylation and bi-galactosylation. It is worth noting that the Schulz et al. study involved a complex genetic engineering strategy where human β4GalT1 was knocked into a ‘safe-harbour’ locus in the CHO genome. Ectopic β4GalT expression has been combined with UMG feeding in order to achieve further mAb β-1,4 galactosylation [3]. This study found that transient overexpression of human β4GalT1 in HEK cells resulted in 70% β-1,4 galactosylation of mAb Fc glycans and a product titre of 0.08 g/L. The authors then supplemented cell culture with D-galactose to achieve 82%β-1,4 galactosylation but observed a reduction in product titre to 0.05 g/L [3].

3. In Vitro Enzymatic β-1,4 Galactosylation

Therapeutic protein β-1,4 galactosylation has also been modified through in vitro treatment of the glycoprotein with bovine β4GalT1. Tayi and Butler [4] achieved increases in mAb Fc β-1,4 galactosylation from 82% to 96% (including 96% bi-galactosylation) for mAb1 and from 39% to 98% (92% bi-galactosylation) for mAb2. Despite presenting the best increases in mono- and bi-β-1,4 galactosylation, in vitro enzymatic glycan remodelling presents two key limitations: (1) In vitro enzymatic β-1,4 galactosylation is extremely expensive at large scales. It requires additional bioprocessing steps and increases the burden on downstream purification to remove the added β4GalT (and contaminants thereof), thus increasing bioprocess capital expenditure. In addition, the in vitro enzymatic processing steps require 24 h to 48 h to achieve high conversions. These additional steps raise bioprocessing costs by increasing overall processing time. Consumable costs, in particular, that of the β4GalT1 enzyme seem prohibitive at large scales. (2) In vitro enzymatic β-1,4 galactosylation requires a complex consumable: the β4GalT enzyme. Bovine β4GalT has been used to perform in vitro β-1,4 galactosylation. However, the use of animal-derived components has been phased out of biopharmaceutical operations over the past 20 years in order to reduce variability and address supply chain limitations. β4GalT1 must be sourced from animal products or through recombinant expression in mammalian cells, because the enzyme itself is glycosylated. This leads to a situation where a glycoprotein consumable must be produced in mammalian cells to produce therapeutic glycoproteins.

What is needed is an improved method of therapeutic protein β-1,4 galactosylation, which overcomes some or all of the limitations of the above current technologies.

According to a first aspect of the present invention, there is provided a cell, wherein the cell is modified, for example to enhance β-1,4 galactosylation of a polypeptide product produced by the cell, the modifications comprising:
reducing O-GalNAc galactosylation activity in the cell by reduction of functional COSMC molecular chaperone in the cell and/or by reduction of functional T-synthase in the cell; and overexpression of β1,4-galactosyltransferase in the cell.

The invention advantageously provides enhanced β-1,4 galactosylation of polypeptide product. In particular, the invention has been demonstrated to maximise the β-1,4 galactosylation of asparagine(N)-linked glycans of therapeutic monoclonal antibodies (mAbs) produced in cells (for example, Chinese Hamster Ovary cells, CHO). The invention combines two genetic modifications to simultaneously eliminate metabolic bottlenecks (by increasing the intracellular availability of UDP-Gal, which is the co-substrate required for (3-1,4 galactosylation) and cellular machinery bottlenecks (by increasing the amount of β4GalT1 expressed by the cells). The invention includes eliminating the expression of COSMC, for example using a specific zinc finger nuclease [5] or CRISPR-Cas9 technology [6]. Abrogation of COSMC expression eliminates threonine/serine(O)-linked 13-1,3 galactosylation, thus yielding cellular glycoproteins bearing the so-called Tn antigen. Eliminating O-linked galactosylation greatly reduces (by approximately 30% [7]) the consumption of uridine diphosphate galactose (UDP-Gal), the donor metabolite required for all galactosylation reactions, towards cellular galactosylation and increases UDP-Gal availability towards N-linked β-1,4 galactosylation of the therapeutic protein product. The invention further includes transfecting cells with the gene for β4GalT. Ectopic expression of the β4GalT enzyme increases the capacity and rate with which the cells add β-1,4-linked galactose residues to glycoprotein N-linked glycans.

Reduced O-GalNAc Galactosylation

In one embodiment, reduced O-GalNAc galactosylation comprises the substantial elimination of O-GalNAc galactosylation activity in the cell. In one embodiment, O-GalNAc galactosylation of polypeptides by the modified cell may not be detectable.

Cellular O-GalNAc galactosylation can be measured by flow cytometry, for example using lectin-aided flow cytometry. VVL (Vicia villosa) lectin binds with high specificity to the Tn-antigen (abrogated O-glycosylation). Cells can be incubated with fluorescently labelled or biotinylated binding agent, such as VVL, and analysed or selected using flow cytometry. The population of cells that give a signal higher than the control for binding agent (e.g. VVL) binding are those where O-linked galactosylation has been abrogated. Cells that present no fluorescence have O-linked galactosylation. Thus, flow cytometry can provide relative quantification of cell surface O-galactosylation. O-galactosylation using VVL flow cytometry may use the method reported by Stolfa et al. (Sci Rep 6, 30392 (2016). https.//doi.org/10.1038/srep30392), which is herein incorporated by reference. 0-glycans reported in CHO cells lines (Yang et al. Molecular & Cellular Proteomics, 2014, 13 (12) 3224-3235. https://doi.org/10.1074/mcp.M114.041541, herein incorporated by reference) are T-antigen, ST-factor and sialyl-Tn antigen, which can be detected with binding agents of Amaranthus caudatus (ACL) or Peanut agglutinin (PNA) and Jacalin lectins, respectively.

O-linked galactosylation can also be measured using conventional liquid chromatography or mass spectrometry-based methods (Mulagapati et al. Biochemistry 2017, 56, 9, 1218-1226. https://doi.org/10.1021/acs.biochem.6b01244, which is herein incorporated by reference).

O-GalNAc galactosylation may be prevented or reduced in the cell by the removal of the ability of the cell to provide functional COSMC molecular chaperone. The cell may be modified such that COSMC molecular chaperone is not expressed (e.g. a COSMC molecular chaperone knockout). Alternatively, the expression of COSMC molecular chaperone may be reduced in the modified cell. In one embodiment, reducing O-GalNAc galactosylation activity in the cell may be by knockout of functional COSMC molecular chaperone in the cell. The knockout may comprise the genetic knockout of the COSMC gene C1GalT1C1 (Gene ID: 2375260). The genetic knockout may comprise an insertion, deletion, or one or more point-mutations in the COSMC gene sequence. In one embodiment, the gene sequence (C1GalT1C1) encoding COSMC molecular chaperone may be deleted or partially deleted. In another embodiment the gene sequence (C1GalT1C1) encoding COSMC molecular chaperone may be modified with a DNA sequence insert, for example to knock out the gene. In one embodiment, the gene sequence (C1GalT1C1) encoding COSMC molecular chaperone may be substituted with a non-functional version thereof.

In another embodiment, translation from the COSMC gene (C1GalT1C1) may be silenced/supressed, for example by RNA silencing. RNA silencing (or RNA interference) refers to a family of gene silencing effects by which gene expression is negatively regulated by non-coding RNAs such as microRNAs. RNA silencing may also be defined as sequence-specific regulation of gene expression triggered by double-stranded RNA (dsRNA). Therefore, in one embodiment, the cell may be contacted with an RNA molecule (such as an miRNA, siRNA or piRNA) capable of targeting the COSMC gene sequence, or transcripts thereof. In another embodiment, the cell may be modified to encode and express such an RNA molecule.

In another embodiment, the COSMC molecular chaperone may be modified such that it is not functional, or substantially reduced in function. The term “functional” is intended to refer to the functional ability of the COSMC molecular chaperone to avoid aggregation, proteasomal degradation and, thus, activity of T-synthase in the Golgi apparatus. The modification may comprise one or more mutations in the COSMC molecular chaperone amino acid sequence relative to wild-type. The mutation may comprise one or more amino acid residue substitutions, deletions or additions. The modification may comprise a truncation of the COSMC molecular chaperone amino acid sequence. The skilled person will be familiar with techniques and sequence modifications that can be made to substantially eliminate or reduce the function of an active protein such as the COSMC molecular chaperone.

In another embodiment, reducing O-GalNAc galactosylation activity in the cell may be by knockout of functional T-synthase in the cell. O-GalNAc galactosylation may be prevented or reduced in the cell by the removal of the ability of the cell to provide functional T-synthase. The T-synthase may be Core 1 synthase, glycoprotein-N-acetylgalactosamine 3-beta-galactosyltransferase, 1 (also known as C1GALT1). In one embodiment, the C1GALT1 is GeneBank number RLQ78471.1 (https://www.ncbi.nlm.nih.gov/protein/RLQ78471.1). In one embodiment, the C1GALT1 is encoded by the C1galt1 gene of Gene ID number 100761169.

The cell may be modified such that T-synthase is not expressed (e.g. a T-synthase knockout). Alternatively, the expression of T-synthase may be reduced in the modified cell. In one embodiment, reducing O-GalNAc galactosylation activity in the cell may be by knockout of functional T-synthase in the cell. The knockout may comprise the genetic knockout of the gene encoding T-synthase. The genetic knockout may comprise an insertion, deletion, or one or more point mutations in the T-synthase gene sequence. In one embodiment, the gene sequence encoding T-synthase may be deleted or partially deleted. In another embodiment the gene sequence encoding T-synthase may be modified with a DNA sequence insert, for example to knock out the gene. In one embodiment, the gene sequence encoding T-synthase may be substituted with a non-functional version thereof.

In another embodiment, translation from the T-synthase gene may be silenced/supressed, for example by RNA silencing. In one embodiment, the cell may be contacted with an RNA molecule (such as an miRNA, siRNA or piRNA) capable of targeting the T-synthase gene sequence, or transcripts thereof. In another embodiment, the cell may be modified to encode and express such an RNA molecule.

In another embodiment, the T-synthase may be modified such that it is not functional, or substantially reduced in function. The term “functional” is intended to refer to the functional ability of T-synthase to carry out O-GalNAc galactosylation. The modification may comprise one or more mutations in the T-synthase amino acid sequence relative to wild-type. The mutation may comprise one or more amino acid residue substitutions, deletions, or additions. The modification may comprise a truncation of the T-synthase amino acid sequence. The skilled person will be familiar with techniques and sequence modifications that can be made to substantially eliminate or reduce the function of an active protein such as the T-synthase.

In one embodiment, the cell has reduced or eliminated levels of functional COSMC molecular chaperone. In one embodiment, the cell has reduced or eliminated levels of functional T-synthase. In one embodiment, the cell has reduced or eliminated levels of functional COSMC molecular chaperone and functional T-synthase. In another embodiment, reducing O-GalNAc galactosylation activity in the cell by knockout of functional COSMC molecular chaperone in the cell and knockout of functional T-synthase in the cell.

The skilled person will be familiar with techniques to genetically modify DNA, for example to eliminate the expression of genes, such as those encoding COSMC and T-synthase. For example a specific zinc finger nuclease [5] or CRISPR-Cas9 technology [6] can be used for specific genetic modification.

The gene knock out of COSMC and/or T synthase may comprise insertion/deletion of one or more bases within the encoding gene. Ronda et al. (2014. Biotechnology and Bioengineering. Vol. 111 (8), pp. 1604-1616. https://doi.org/10.1002/bit.25233; incorporated herein by reference) describes an indel (insertion/deletion) strategy that can be used to knock out genes such as COSMC.

In an alternative embodiment, the reduction of functional T-synthase in the cell may comprise inhibition of T-synthase in the cell. T-synthase may be inhibited with an agent arranged to block T-synthase activity, such as a synthetic GalNAc sugar ligand arranged to block the active site of the T-synthase. T-synthase may be inhibited by providing GalNAc sugars having 3- or 4-hydroxyl groups removed. Such modified GalNAc sugars are known to inhibit C1GALT1 activity as reported by Brockhausen et al. (Biochemistry and Cell Biology, 1992, 70(2): 99-108, (https://doi.org/10.1139/o92-015), which is herein incorporated by reference.

Overexpression of β1,4-galactosyltransferase

In one embodiment the overexpression of β1,4-galactosyltransferase in the cell may be provided by providing the ectopic expression of a β1,4-galactosyltransferase I. In one embodiment, the cell is transformed with nucleic acid encoding β1,4-galactosyltransferase. The nucleic acid sequence encoding the β1,4-galactosyltransferase I may be chromosomally integrated (stably transformed). In another embodiment, the β1,4-galactosyltransferase may be overexpressed from a plasmid transformed into the cell (e.g. transient expression).

The skilled person will recognise that the β1,4-galactosyltransferase is functional with β1,4-galactosyltransferase activity. The β1,4-galactosyltransferase may be a recombinant β1,4-galactosyltransferase. The β1,4-galactosyltransferase may be a heterologous β1,4-galactosyltransferase. In one embodiment, the β1,4-galactosyltransferase is a mammalian, preferably a human, β1,4-galactosyltransferase. The β1,4-galactosyltransferase may comprise the sequence of SEQ ID NO: 1 (NCBI Reference Sequence: NP_001488.2), or a variant thereof having functional β1,4-galactosyltransferase activity.

In one embodiment, the β1,4-galactosyltransferase is β1,4-galactosyltransferase isoform I (β4GalT1). Other β1,4-galactosyltransferase isoforms may be contemplated by the skilled person, such as any of isoforms 1-7. Preferably, the β1,4-galactosyltransferase is an isoform selected from isoforms 1, 2, 3 and 4 (NCBI Reference Sequences: NP_001488.2, NP_001365424, NP_001365425.1, and NP_001365426.1 respectively). The skilled person will recognise that the β1,4-galactosyltransferase may be sourced from different organisms, such as Homo sapiens, Mus musculus, Rattus novergicus, Pan troglodytes, and Bos taurus. Preference is given to human β4GalT1 (UniProtKB protein P15291), as this particular enzyme has been reported to achieve the highest levels of β-1,4 galactosylation in CHO-derived glycoproteins.

The β1,4-galactosyltransferase may be overexpressed, for example relative to the normal expression of an unmodified cell (for example under the same conditions). The expression/overexpression of β1,4-galactosyltransferase may be at least a two-fold increase in expression relative to the typical expression of β1,4-galactosyltransferase in an unmodified cell under the same cell culture conditions. The expression/overexpression of β1,4-galactosyltransferase may be at least a 3, 4, 5- or 10-fold increase in expression relative to the typical expression of β1,4-galactosyltransferase in an unmodified cell under the same cell culture conditions. The increase in expression may be sufficient to provide an increase in β1,4 galactosylation of the polypeptide product relative to an unmodified cell. Successful overexpression of β1,4-galactosyltransferase may be detectable by detecting an increase in β1,4 galactosylation of the polypeptide product.

The overexpression may be inducible or constitutive. In one embodiment, the expression of β1,4-galactosyltransferase is constitutive. The control of the expression may be provided by a promoter, which may be a constitutive or inducible promoter in the cell. In one embodiment, the cell is transformed with nucleic acid encoding β1,4-galactosyltransferase under the control of a promoter. The promoter may be ectopic to the cell. Typical promoters that may be used include any promoter that induces overexpression of the β1,4-galactosyltransferase in the cell, such as a promoter selected from CMV, SV40, PGK-1, Ubc and CAG. In one embodiment the promoter of the β1,4-galactosyltransferase is human Elongation Factor-1α (hEF-1α) core promoter. β1,4-galactosyltransferase may be induced by a composite hEF1-HTLV promoter, that couples the human Elongation Factor-1α (EF-1α) core promoter and the R segment and part of the U5 sequence (R-U5′) of the Human T-Cell Leukemia Virus (HTLV) Type 1 Long Terminal Repeat.

In an alternative embodiment, the cell may be transformed with nucleic acid encoding β1,4-galactosyltransferase where it is chromosomally integrated to be under the control of an endogenous promoter of the cell. For example, an endogenous promoter that promotes constitutive expression.

In another embodiment, the endogenous β1,4-galactosyltransferase of the cell may be overexpressed. For example, an ectopic promoter may be transformed and inserted into the chromosome of the cell to control the expression of β1,4-galactosyltransferase. In one embodiment, the endogenous β1,4-galactosyltransferase promoter is replaced by recombination with a constitutive or inducible promoter, such as TetR-CMV promoter that is regulated by doxycycline.

In one embodiment, the nucleic acid transformed into the cell is a plasmid encoding the β1,4-galactosyltransferase and/or promoter. The plasmid may comprise a sequence encoding the β1,4-galactosyltransferase and/or promoter and flanking sequences of the chromosomal DNA for heterologous recombination of the sequence encoding the β1,4-galactosyltransferase and/or promoter into the chromosome of the cell. In one embodiment, the plasmid encoding the β1,4-galactosyltransferase and promoter comprises pUNO (e.g. from InvivoGen). pUNO provides the hb4GalT1 gene and uses a blasticidin resistance gene. Blasticidin allows for quick selection of transfected cells.

The Cell

The cell may be a eukaryote. The cell may be a mammalian cell. In another embodiment, the cell may be selected from a mammalian cell, insect cell, and protozoan cells, such as Leishmania tarentolae. In one embodiment, the cell is suitable for production of a polypeptide product. Such cells may be selected from CHO, NS0, SP2/0, PER.C6, Sf9, VERY, BH, HeLa, COS, MDCK, 293, 293T, 3T3, WI38, BT483, Hs578T, HTB2, BT20, T47D, CRL7030 and Hs578Bst. Mammalian cells may be selected from CHO, NS0, SP2/0, PER.C6, VERY, BH, HeLa, COS, MDCK, 293, 293T, 3T3, WI38, BT483, Hs578T, HTB2, BT20, T47D, CRL7030 and Hs578Bst. The CHO cell may be a CHO cell variant, for example selected from the group comprising DG44, CHO-S, CHO-K1, CHO-DXB11, and GS-CHO (CHO-K1-derived cell line employing the glutamine synthetase (GS) gene expression system) variants.

In one embodiment the cell is a CHO cell (Chinese Hamster Ovary cell) or a HEK cell (Human Embryonic Kidney cell), such as HEK-293. In a preferred embodiment, the cell is a CHO cell. The CHO cell may be of the CHO cell line CHO VRC01 or CHO DP12. The skilled person will recognise that any appropriate cell may be used/modified which has the same or substantially similar glycosylation pathways as CHO cells.

In embodiment wherein the cell is an insect cell, the cell may comprise or consist of Sf9. The insect cell may be a lepidopteran cell.

The Polypeptide Product

The polypeptide product may be a heterologous polypeptide. The polypeptide product may be a recombinant polypeptide. The polypeptide product may comprise a physiologically or metabolically relevant protein. The polypeptide product may comprise a bacterial, or bacterially derived protein. The polypeptide product may comprise a mammalian, or mammalian derived protein. The polypeptide product may be any peptide, polypeptide or protein. The polypeptide product may comprise research, diagnostic or therapeutic molecules.

In one embodiment the polypeptide product is an antibody peptide, such as one or more peptides of a monoclonal antibody. In one embodiment, the polypeptide product is an antibody or a fragment thereof, such as a heavy or light chain peptide.

The polypeptide product may comprise an enzyme or substrate thereof, a protease, an enzyme activity modulator, a peptide aptamer, an antibody, a modulator of protein-protein interaction, a growth factor, or a differentiation factor.

The polypeptide product may be selected from any of the group comprising a therapeutic molecule; a drug; a pro-drug; a functional protein or peptide, such as an enzyme or a transcription factor; a microbial protein or peptide; and a toxin. The polypeptide product may comprise a viral particle protein.

The polypeptide product may be between about 20 and about 30,000 amino acids in length. The polypeptide product may be between about 20 and about 10,000 amino acids in length. The polypeptide product may be between about 20 and about 5,000 amino acids in length. The polypeptide product may be between about 20 and about 1000 amino acids in length. The polypeptide product may be at least about 20 amino acids in length. The polypeptide product may be at least about 100 amino acids in length.

The polypeptide product may comprise one or more, or all the peptides of anti-IL-8 IgG1κ MAb. The polypeptide product may comprise one or more, or all the peptides of a therapeutic selected from the group comprising [fam-]trastuzumab deruxtecan, Leronlimab, Narsoplimab, REGNEB3, Sacituzumab govitecan, Tafasitamab, Inebilizumab, Satralizumab, Eptinezumab, Isatuximab, Teprotumumab, Crizanlizumab, Enfortumab vedotin, Polatuzumab vedotin, Risankizumab, Romosozumab, Burosumab, Cemiplimab, Emapalumab, emapalumab-lzsg, Erenumab, Fremanezumab, Galcanezumab, Gemtuzumab ozogamicin, Ibalizumab, ibalizumab-uiyk, Lanadelumab, Mogamulizumab, Ravulizumab (ALXN1210), Tildrakizumab, Avelumab, Benralizumab, Brodalumab, Dupilumab, Durvalumab, Emicizumab, Guselkumab, Inotuzumab ozogamicin, Ocrelizumab, Sarilumab, Atezolizumab, Bezlotoxumab, Ixekizumab, Obiltoxaximab, Olaratumab, Reslizumab, Alirocumab, Daratumumab, Dinutuximab, Elotuzumab, Evolocumab, Mepolizumab, Necitumumab, Secukinumab, Nivolumab, Pembrolizumab, Ramucirumab, Siltuximab, Vedolizumab, Alemtuzumab, Obinutuzumab, Ado-trastuzumab emtansine, Pertuzumab, Raxibacumab, Belimumab, Brentuximab vedotin, Ipilimumab, Denosumab, Canakinumab, Golimumab, Ofatumumab, Tocilizumab, Ustekinumab, Eculizumab, Panitumumab, Bevacizumab, Cetuximab, Natalizumab, Omalizumab, Adalimumab, Ibritumomab tiuxetan, Basiliximab, Infliximab, Palivizumab, Trastuzumab, Rituximab, and Abciximab.

The polypeptide product may benefit any glycoprotein that does not have O-linked glycans. In particular, mAbs in clinical trials or already on the market could benefit from the invention due to (i) increased homogeneity, (ii) enhanced cytotoxic effector functions (ADCC, CDC, ADCP) for oncolytic mAbs and (iii) increased sialylation for immune response modulation in anti-inflammatory mAbs.

In one embodiment the polypeptide product is a viral protein. The viral protein may comprise a component of a viral vector such as rAAV. The rAAV may be AAV2 or AAV5 serotypes.

rAAV vectors, and other viral vectors, are used for gene therapy. rAAV5 has at least one N-linked glycosylation site that could benefit from the invention because rAAV immunogenicity and target cell interactions are predicted to be mediated by O-linked glycans.

In one embodiment, a plurality of products may be expressed, or encoded for expression, by the cell. The plurality of products may comprise the heavy and light chains of an antibody or antibody variant molecule.

The cell may or may not be transformed with the nucleic acid encoding the polypeptide product. For example, the cell may be modified according to the invention and may be capable of being transformed with nucleic acid encoding a polypeptide product (e.g. the cell maybe modified later to express the polypeptide product of choice). In another embodiment, the cell is further modified to express the polypeptide product. In one embodiment the cell has been transformed with nucleic acid encoding the polypeptide product.

The nucleic acid encoding the polypeptide product may be stably transformed (chromosomally integrated) or transiently transfected (e.g. on an expression plasmid). The skilled person will be familiar with techniques and publicly available technologies to transform a cell to cause expression of a desired polypeptide product. For example, the polypeptide product may be encoded on an expression plasmid, construct or viral vector (such as lentiviral vector) suitable for transformation and expression of the polypeptide product in the cell. The expression plasmid or construct may comprise a promoter operably linked to the gene encoding the polypeptide product. The promoter may be inducible or constitutive. The plasmid or construct may be arranged to integrate into the chromosome of the cell, for example by homologous recombination or insertional elements. The plasmid or construct may comprise a selection marker to determine successfully transformed cells.

The modification of the invention can be applied to cells where the recombinant product is expressed via any selection/amplification strategy. The stably transfected cell lines may be based on a gene amplification and expression system (e.g. DHFR) or on a metabolic selection (e.g. glutamine synthetase).

Molecular components of the plasmid or construct may include one or more of an origin of replication, promoter and enhancer element (natural or synthetic), a late promoter, introns, termination signals (poly adenylation), viral amplifiers, IRES elements, and a selectable gene for propagation.

Other elements of the plasmid or construct may include one or more of: genetic regulators as WPRE (Woodchuck Hepatitis Virus (WHP) Post-transcriptional Regulatory Element) sequences, scaffold/matric attachment regions (S/MARs), locus control regions (LCRs), insulators as ubiquitous chromatin opening elements (UCOEs), and stabilizing anti-repressor elements (STAR).

The nucleic acid, for example encoding the polypeptide product and/or β1,4-galactosyltransferase, may be integrated into the chromosome of the cell via recombination sites for example using Cre recombinase, FLP-FRT or phiC31 integrase.

Positive selection markers may be suitable for CHO cells, such as those that rely on selection with Zeocin, Puromycin, Hygromycin B or G418.

In one embodiment, the cell may be further modified to express α-2,6 Sialyltransferase (α6SiaT) to enhance the content of N-acetylneuraminic acid (Neu5Ac) on a polypeptide product, such as mAb N-linked glycans. The α-2,6 Sialyltransferase (α6SiaT) may be arranged to be overexpressed in the cell. For example the cell may further comprise nucleic acid encoding α-2,6 Sialyltransferase (α6SiaT), which may be heterologous to the cell. The nucleic acid encoding α-2,6 Sialyltransferase (α6SiaT) may be chromosomally integrated or extra-chromosomal, such as on an expression plasmid.

Other Aspects

According to another aspect of the present invention, there is provided a method of modifying a cell, wherein the cell is modified to enhance β-1,4 galactosylation of a polypeptide product, the modifications comprising:
reducing O-GalNAc galactosylation activity in the cell by reduction of functional COSMC molecular chaperone in the cell and/or by reduction of functional T-synthase in the cell; and
modification of the cell to provide overexpression of β1,4-galactosyltransferase in the cell.

The method may comprise transforming the cell with nucleic acid encoding the β1,4-galactosyltransferase.

Additionally, the method may comprise modifying the gene(s) encoding the COSMC molecular chaperone and/or T-synthase, or modifying regulatory elements thereof. The modification may be a knockout of the gene(s). In one embodiment, the gene(s) are deleted, or disrupted with an insertion. In another embodiment, the nucleic acid sequence of the gene(s) is modified, such that the COSMC molecular chaperone and/or T-synthase is not expressed in a functional form, or expressed in a form with a significant reduction in functional activity/ability.

The method may further comprise modifying the cell to express a polypeptide product. For example, the cell may be transformed with nucleic acid encoding the polypeptide product for expression.

Transformed nucleic acid may comprise a selection marker for identifying successfully transformed cells. Therefore, the method may further comprise the step of selecting the successfully transformed cells, which have been modified. The selection may be carried out by growth on selecting media or in the presence of an agent to allow for selection.

The modifications to the cell may be provided sequentially, or in one transformation event.

According to another aspect of the present invention, there is provided a method of producing a polypeptide product, the method comprising obtaining or providing a cell modified according to the invention herein, and culturing the cell under conditions suitable for expression of the polypeptide product.

The polypeptide product may be produced to have at least 80% β-1,4 galactosylation and/or at least 50% bi-galactosylated. In another embodiment, the polypeptide product may be produced to have at least 90% β-1,4 galactosylation and/or at least 60% bi-galactosylated. In another embodiment, the polypeptide product may be produced to have at least 95% β-1,4 galactosylation and/or at least 75% bi-galactosylated.

In another embodiment, the polypeptide product may be produced to have at least 0.5 fold increased β-1,4 galactosylation and/or at least 0.5 fold increased bi-galactosylation. In another embodiment, the polypeptide product may be produced to have at least 2 fold increased β-1,4 galactosylation and/or at least 2 fold increased bi-galactosylation. In another embodiment, the polypeptide product may be produced to have at least 5 fold increased β-1,4 galactosylation and/or at least 5 fold increased bi-galactosylation. The fold increase may be relative to the same cell that has not been modified according to the invention, for example under the same culture conditions.

Culturing the cell under conditions suitable for expression of the polypeptide product may comprise culturing the cell in cell growth media at a temperature and atmosphere suitable for growth of the cell, such as standard conditions (e.g. about 37° C. and about 5% CO₂ for example for mammalian cells). The skilled person will be familiar with suitable growth medias for the cell to be maintained and grow. For example, the cell growth media may comprise basal media, such as MEM (Minimum Essential Medium) or DMEM (Dulbecco's Modified Eagle's Medium), complex media such as IMDM (Iscove's Modified Dulbecco's Medium) or RPMI-1640, or serum-free media such as Ham's F-10 or F-12. The basal media may comprise chemically defined basal media. The cell culture may be supplemented with cell feed.

Additional glucose and/or amino acids such as L-glutamine, may be added during the cell culture. Insect cells may be grown in suitable insect cell growth media, such as Grace's media.

The culture may be a continuous culture or a batch culture, such as a fed-batch culture. In one embodiment, the culture is a fed-batch culture.

In one embodiment, the cells may be cultured with a UMG feeding strategy (i.e. addition of uridine/manganese/galactose in the media).

Advantageously, coupling with UMG feeding strategies could achieve even higher levels of galactosylation. The cells would require far less dosing relative to standard UMG feeding because most of the UMG fed would bypass cellular O-galactosylation and go directly into N-galactosylation.

The polypeptide product may be harvested from the cells and/or supernatant. For example, the polypeptide product may be isolated and purified from the remaining cell and/or media content. The skilled person will be familiar with a range of suitable purification technologies and techniques for purification of polypeptide product from cells such as mammalian cells.

According to another aspect of the present invention, there is provided a polypeptide produced by the modified cell of the invention herein, or produced by the method of the invention herein.

According to another aspect of the present invention, there is provided the use of a modified cell according to the invention herein for the production of a polypeptide product.

According to another aspect of the present invention, there is provided nucleic acid, such as a plasmid, encoding:
a genetic modification element(s) that is targeted to knock-out or reduce functional COSMC molecular chaperone and/or T-synthase expression in a cell;
b4GalT for expression in the cell; and
   optionally a selection marker, such as an antibiotic resistance gene.

The plasmid may further encode a polypeptide product for expression.

According to another aspect of the present invention, there is provided a kit comprising:
a first plasmid encoding:
a genetic modification element(s) that is targeted to knock-out or reduce functional COSMC molecular chaperone and/or T-synthase expression in a cell; and
a second plasmid encoding b4GalT for expression in a cell and optionally a selection marker, such as an antibiotic resistance gene.

The kit may further comprise a plasmid encoding a polypeptide product for expression, for example as described herein.

The kit may further comprise cells, for example for transduction and genetic modification with the plasmids. The cells may be cells as described herein, such as mammalian cells (e.g. CHO cells). The kit may further comprise one or more buffers and/or reagents for transformation of the cells. The kit may comprise one or more selection agents for selection of successfully transformed or modified cells.

In one embodiment, the cells may be capable of expressing, or may have been previously modified to express, the polypeptide product.

The genetic modification element(s) may comprise DNA for insertion into the cell's chromosome. Additionally or alternatively the genetic modification elements may provide guide RNA and/or a nuclease. The genetic modification element(s) may comprise zinc finger nuclease, TALEN (transcription activator-like effector nuclease) or guide RNA (gRNA) and Cas9 (for CRISPR modification). The genetic modification element(s) may comprise sequences arranged to insert (for example by double recombination) and disrupt the targeted genetic elements of COSMC molecular chaperone and/or T-synthase in the cell. The genetic modification element(s) may comprise silencing RNA sequences (e.g. miRNA or siRNA) arranged to bind to transcripts of genes encoding COSMC molecular chaperone and/or T-synthase. The genetic modification element(s) may comprise homologous recombination cassettes with section markers, such as antibiotic resistance genes.

### Definitions

The term “enhance β-1,4 galactosylation of the polypeptide product” is understood to mean that the polypeptide product is β-1,4 galactosylated in the cell. In one embodiment, the β-1,4 galactosylation of the polypeptide product is greater than it would have been in the unmodified cell, such as a wild-type cell. In one embodiment, the β-1,4 galactosylation of the polypeptide product is greater than it would have been in the same cell type that does not reduce O-GalNAc galactosylation activity and/or does not overexpress β1,4-galactosyltransferase in the cell, such as a wild-type cell.

The β-1,4 galactosylation may be the β-1,4 galactosylation of asparagine(N)-linked glycans. Enhanced β-1,4 galactosylation may comprise an increase in product β-1,4 galactosylation to at least 80%, and/or increase in bi-galactosylated species to at least 50%.

By “antibody” we include substantially intact antibody molecules, as well as chimeric antibodies, human antibodies, humanised antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments and derivatives of the same. In particular, the term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to as a “mAb”.

It has been shown that fragments of a whole antibody can perform the function of binding antigens. Examples of binding fragments of the invention are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers (PCT/US92/09965, incorporated herein by reference) and; (ix) “diabodies”, multivalent or multispecific fragments constructed by gene fusion (WO94/13804, incorporated herein by reference).

Where reference is made to a variant polypeptide or nucleotide sequence, the skilled person will understand that one or more amino acid residue or nucleotide substitutions, deletions or additions, may be tolerated, optionally two substitutions may be tolerated in the sequence, such that it maintains its function. The skilled person will appreciate that 1, 2, 3, 4, 5 or more amino acid residues or nucleotides may be substituted, added or removed without affecting function References to sequence identity may be determined by BLAST sequence alignment (www.ncbi.nlm.nih.gov/BLAST/) using standard/default parameters. For example, the sequence may have at least 99% identity and still function according to the invention. In other embodiments, the sequence may have at least 98% identity and still function according to the invention. In another embodiment, the sequence may have at least 95% identity and still function according to the invention. In another embodiment, the sequence may have at least 90%, 85%, or 80% identity and still function according to the invention. In one embodiment, the variation and sequence identity may be according the full-length sequence. In other embodiments, the variation may be limited to non-conserved sequences and/or sequences outside of active sites, such as binding domains. Therefore, an active site or binding site of a protein may be 100% identical, whereas the flanking sequences may comprise the stated variations in identity. Such variants may be termed “conserved active site variants”.

Amino acid substitutions may be conservative substitutions. For example, a modified residue may comprise substantially similar properties as the wild-type substituted residue. For example, a substituted residue may comprise substantially similar or equal charge or hydrophobicity as the wild-type substituted residue. For example, a substituted residue may comprise substantially similar molecular weight or steric bulk as the wild-type substituted residue. With reference to “variant” nucleic acid sequences, the skilled person will appreciate that 1, 2, 3, 4, 5 or more codons may be substituted, added, or removed without affecting function. For example, conservative substitutions may be considered.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

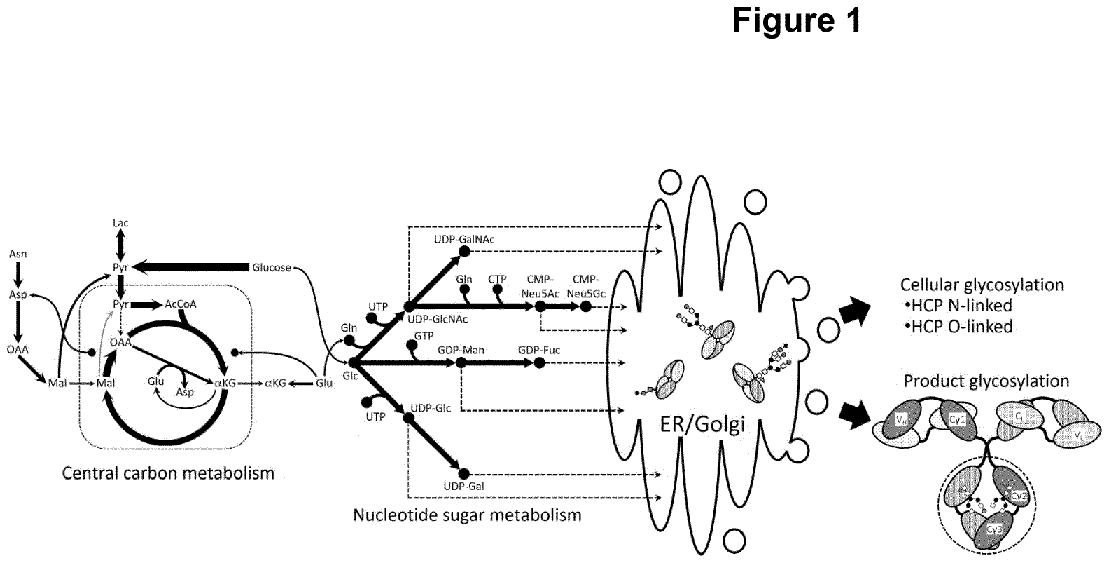

FIG. 1—Sources of mAb N-glycan variability. Variability arises from the glycosylation process. Mechanisms: residence time in Golgi (qₚ)¹, enzyme activity, enzyme accessibility, etc. Metabolism: Nucleotide Sugar Donor (NSD) availability². NSDs are simultaneously consumed for cellular & product glycosylation.

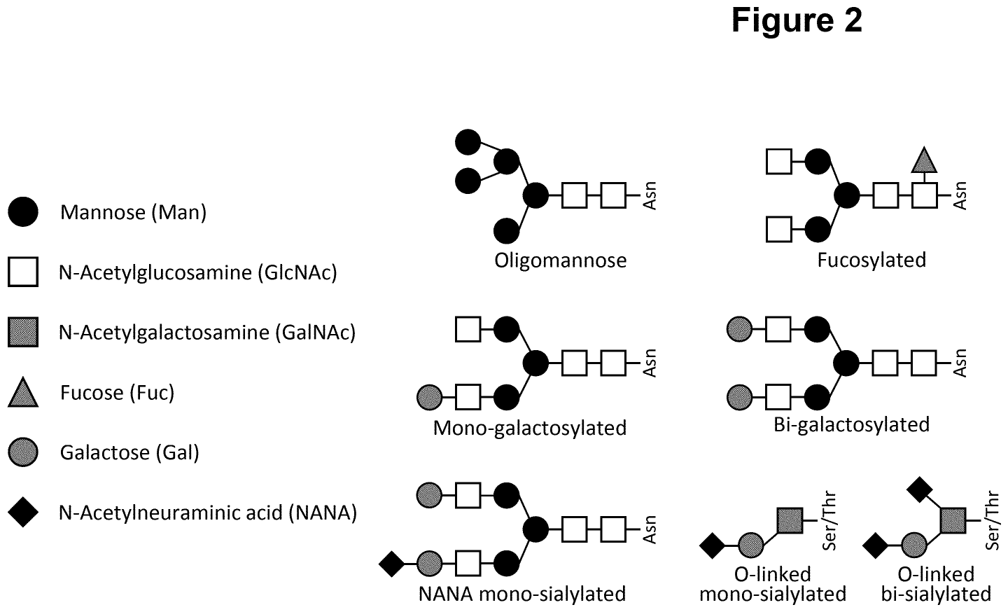

FIG. 2—N-glycan variability.

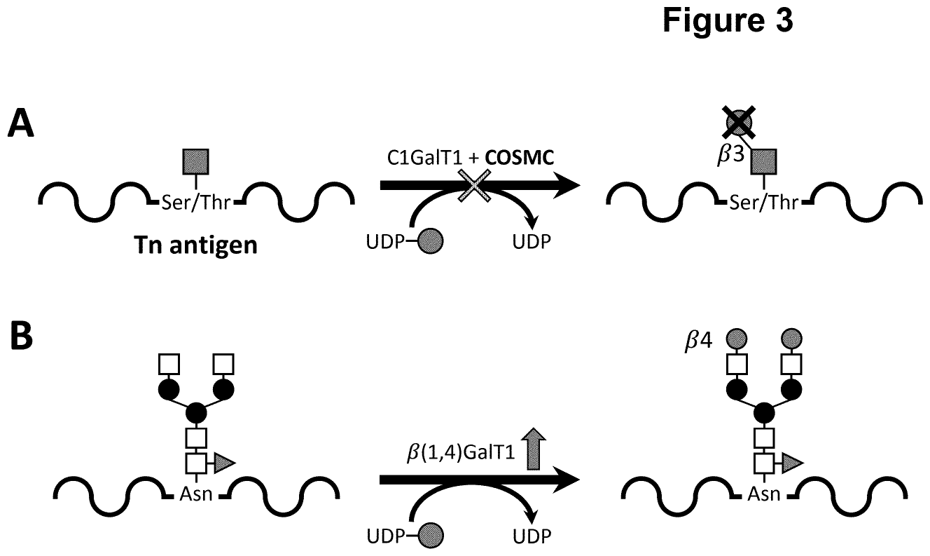

FIG. 3—Increase the mAb galactosylation capacity of CHO cells by: A. Eliminating galactosylation by knocking out the COSMC molecular chaperone; and B. Increasing cellular galactosylation capacity by overexpressing β1,4-galactosyltransferase I.

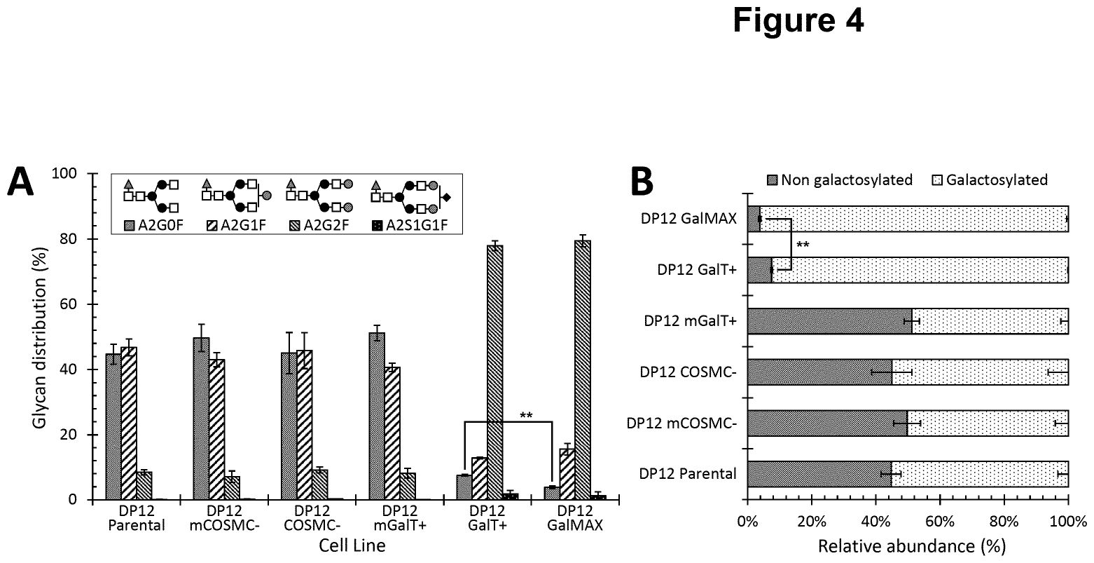

FIG. 4—CHO DP12 Cell Line. Both cell engineering events are required.

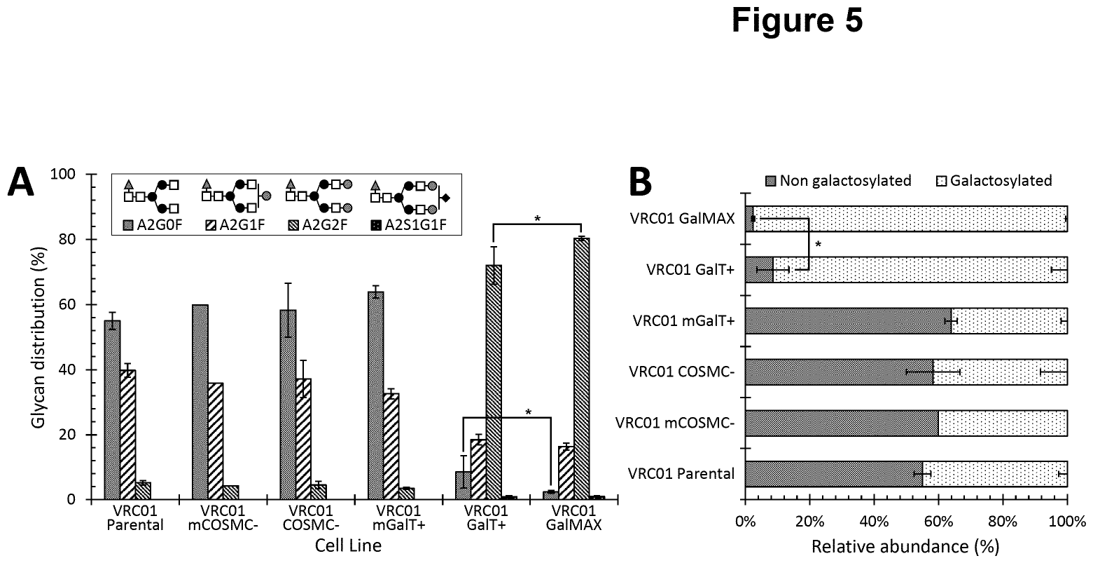

FIG. 5—CHO VRC01 Cell Line. Both cell engineering events are required.

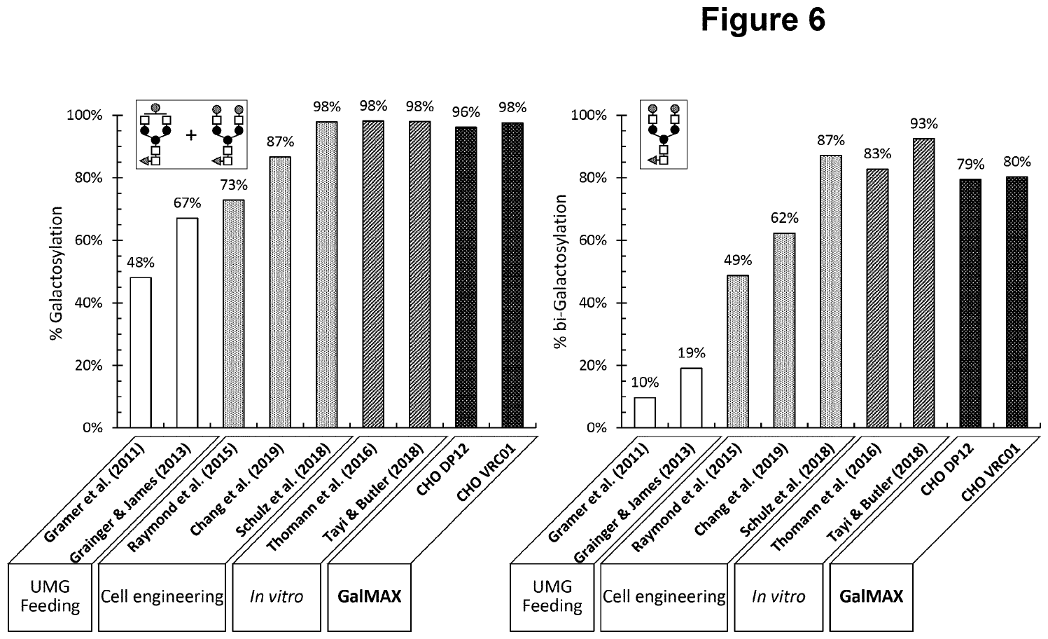

FIG. 6—Comparison with other technologies.

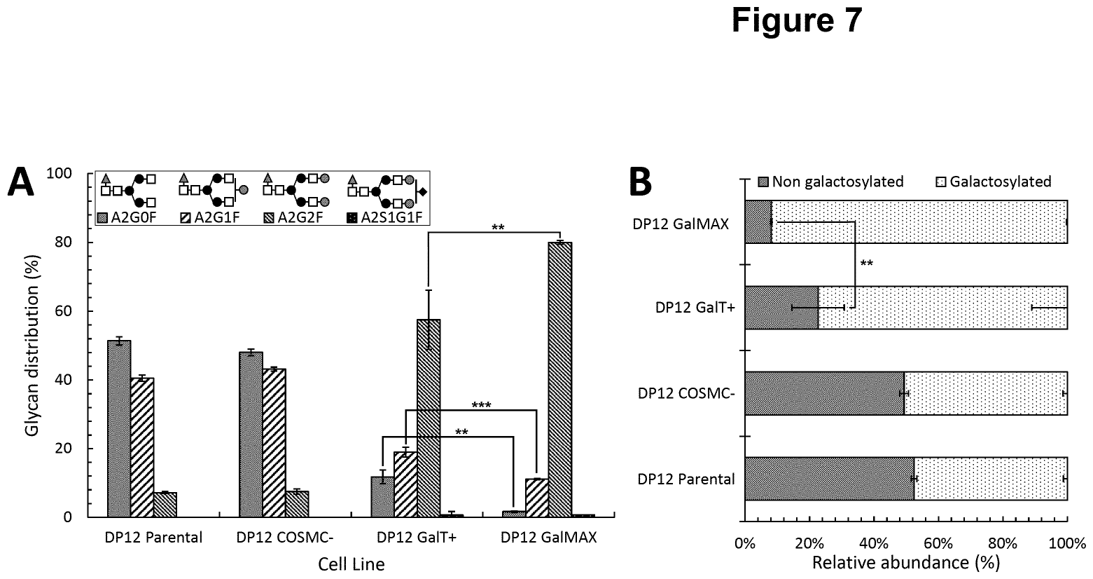

FIG. 7—Fed-batch CHO DP12 cell culture. GalMAX enhances galactosylation

### Example 1—GalMAX—Maximising Therapeutic Protein Galactosylation: Simultaneous Removal of Metabolic and Cellular Machinery Bottlenecks

### SUMMARY

The positive impact of β-1,4 galactosylation on therapeutic protein efficacy has only been reported recently. Table 1 summarises the effects of various glycan modifications on mAb cancer killing ability, and highlights the positive impact galactosylation on safety, CDC, ADCC and PK/PD.

TABLE 1
mAb cancer killing ability. (1,4)-Galactosylation is the source
of therapeutic protein variability. Galactosylation increases
efficacy and reduces heterogeneity (see Raju et al (2012) mAbs
4(3): 385-391 Planinc et al. (2017) Eur. J. Hosp. Pharm. Sci.
Pract. 24(5): 286-292, which is herein incorporated by reference).

| Glycan | Safety | CDC | ADCC | PK/PD |
|---|---|---|---|---|
| Oligomannose | — | Negative | Positive and | Negative |
| | | impact | negative | impact |
| | | | | No impact |
| Fucose | — | No impact | Highly | No impact |
| | | | negative | |
| | | | impact | |
| Galactose | Positive | Positive | Positive | Positive |
| | impact | impact | impact | impact |
| Bisected | — | — | Positive | — |
| | | | impact | |
| NANA | Positive | — | Negative | Positive |
| sialylated | impact | | impact | impact |
| NGNA | Highly negative | — | — | Positive |
| sialylated | impact | | | impact |
| αGal epitope | Highly negative | — | — | Negative |
| | impact | | | impact |

The invention maximises the β-1,4 galactosylation of asparagine(N)-linked glycans of therapeutic monoclonal antibodies (mAbs) produced in cells (such as Chinese Hamster Ovary cells, CHO). The invention combines two genetic modifications to simultaneously eliminate metabolic bottlenecks (by increasing the intracellular availability of UDP-Gal, which is the co-substrate required for β-1,4 galactosylation) and cellular machinery bottlenecks (by increasing the amount of β4GalT1 expressed by the cells). The invention comprises the following two genetic engineering events, which can be performed sequentially or simultaneously:

1. Genetic knockout of Core 1 β3GalT specific molecular chaperone (COSMC).

The first genetic engineering event involves eliminating the expression of COSMC, using a specific zinc finger nuclease [1] or CRISPR-Cas9 technology [2]. Abrogation of COSMC expression eliminates threonine/serine(O)-linked β-1,3 galactosylation, thus yielding cellular glycoproteins bearing the so-called Tn antigen. Eliminating O-linked galactosylation greatly reduces (by approximately 30% [3]) the consumption of uridine diphosphate galactose (UDP-Gal), the donor metabolite required for all galactosylation reactions, towards cellular galactosylation and increases UDP-Gal availability towards N-linked β-1,4 galactosylation of the therapeutic protein product.

2. Genetic overexpression of the β-1,4 galactosyltransferase (β4GalT) enzyme.

The second genetic engineering event involves stably transfecting cells with the gene for β4GalT, which can be of any isotype (1 through 7) and sourced from different organisms (Homo sapiens, Mus musculus, Rattus novergicus, Pan troglodytes, Bos taurus, etc.). Preference is given to human β4GalT1 (UniProtKB protein P15291), as this particular enzyme has been reported to achieve the highest levels of β-1,4 galactosylation in CHO-derived glycoproteins [4].

Ectopic expression of the β4GalT enzyme increases the capacity and rate with which the cells add β-1,4-linked galactose residues to glycoprotein N-linked glycans.

Materials

The CHO DP-12 clone #1934 [aIL8.92 NB 28605/14] (ATCC® CRL-12445™) producing an anti-IL-8 IgG1κ was used for our preliminary studies. This cell line was adapted to grow in suspension using Ex-Cell® 302 serum-free media (Sigma-Aldrich, Cat. No. 14324C) with 4 mM of L-glutamine and 200 nM of MTX. Fed-batch culture was performed by supplementing the basal media (Sigma-Aldrich, Cat. No. 14324C) with 6.25% v/v of Ex-Cell Advanced CHO feed with glucose (Sigma-Aldrich, Cat. No. 24367C) every 48 hours, starting from day 3. The feed was further supplemented with 45% w/v glucose to maintain residual glucose concentration above 4 g/L after feeding. A second CHO cell line (VRC01), which is derived from CHO-K1 and produces a human IgG1κ, was used to confirm the GalMAX strategy. This cell line was adapted for suspension growth in serum-free ActiPro culture media (HyClone Cat. No. SH31039.02) supplemented with 4 mM L-glutamine and 100 nM MTX.

Ectopic expression of human β4GalT1 was performed by transfecting the DP-12 and VRC01 CHO cells with the pUNO1 plasmid bearing the coding sequence of human β4GalT1 (Invivogen Cat. Code puno1-hb4galt1) using an Amaxa® CLB-Transfection Device and an Amaxa® CLB-Transfection Kit (Lonza, Cat. No. VECA-1001), as per the manufacturer's instructions. The pUNO1 scaffold also contains the coding sequence for a blasticidin resistance gene, which was used to select successfully transfected cells.

Knocking out of COSMC was performed using the CRISPR-Cas9 genome editing system using an all-in-one pX458 plasmid (pSpCas9(BB)-2A-GFP) obtained from Addgene (plasmid #48138) [8]. The gRNA sequence (GAATATGTGAGTGTGGATGGAGG) targeting the C1GalT1C1 gene (Gene ID: 2375260) was designed using the CHO Cas9 Target Finder (http://staff.biosustain.dtu.dk/laeb/crispy, target ID 2375260) [6].

Cells successfully transfected with the pX458+sgRNA plasmid were selected for eGFP fluorescence using a FACSAria III Cell Sorter (Beckton-Dickinson) with a 488 nm (blue) excitation laser. Homogeneous populations were gated using forward and side scatter criteria to select singlets. Fluorescent-positive cells were identified using non-transfected cells as the auto-fluorescence control. Cell pools were recovered and cultured. After two weeks, the population of GFP-negative cells were isolated and cultured for an additional three days, after which, cells bearing the COSMC knockout phenotype (bearing only exposed GalNAc residues—the Tn antigen—on their surface O-linked glycans) were identified and isolated through lectin-aided cell sorting using the FACSAria III instrument.

The FITC-labelled Vicia villosa lectin (Vector Labs, Cat. No. B-1235), which is specific for the Tn antigen, was used for COSMC knockout cell sorting. A similar procedure was performed for cells transfected with human β4GalT1, but the lectin used was (fluorescein labelled lectin from Erythrina cristagalli, Vector Labs, Cat. No. FL-1141) to select cells presenting enhanced cell surface β-1,4-galactosylated glycans.

mAb glycan analysis was performed using a recently-developed method that has been optimised for quantification of glycopeptides (Carillo et al. Journal of Pharmaceutical Analysis. Volume 10, Issue 1, February 2020, Pages 23-34. https://doi.org/10.1016/j.jpha.2019.11.008, which is herein incorporated by reference).

Study Results

We have performed proof-of-concept studies in mAb-producing CHO cells where the invention yielded an increase in product β-1,4 galactosylation from 55.2% to 96.2% (CHO DP12) and from 45% to 97.6% (CHO VRC01), including a substantial increase in bi-galactosylated species from 8.4% to 79.4% (CHO DP12) and 5.2% to 80.3% (CHO VRC01) (Table 8 and FIGS. 4 and 5). A key finding confirming the rationale underlying the designed technology is that the simultaneous knock out of COSMC and ectopic expression of human βGalT1 are required to achieve the exceptional increases in product β-1,4 galactosylation obtained with the invention.

To confirm the advantages of the technology, CHO-DP12 was cultured under fed-batch operation where the invention yielded an increase in product β-1,4 galactosylation from 47.7% to 91.8%, including a substantial increase in bi-galactosylated species from 7.2% to 80.0% (Table 9 and FIG. 7). The two key findings are that (i) the invention produces enhanced product β-1,4 galactosylation under fed-batch culture, which is standard practice for large scale mAb manufacturing and (ii) that the invention yields substantially higher galactosylation than the cells overexpressing only β4GalT1, thus indicating that both genetic modifications are required to maximise product β-1,4 galactosylation.

This invention provides a simple and robust solution for key Quality Assurance challenges that arise during the manufacture of recombinant therapeutic glycoproteins, including monoclonal antibodies (mAbs). The key challenges tackled by the invention are those associated (1) the heterogeneity, (2) the therapeutic efficacy (pharmacodynamics) and (3) the serum half-life (pharmacokinetics) of therapeutic glycoproteins, as outlined below.

1. Maximising β-1,4 Galactosylation Reduces the Heterogeneity of Therapeutic Proteins.

β-1,4 galactosylation is the major source of therapeutic protein variability [10, 11] and arises from even subtle process deviations during manufacturing operations [12]. Nutrient availability, metabolite accumulation, temperature, pH and other bioprocess conditions vary during the large-scale cell culture processes whereby therapeutic proteins are manufactured. Variations in bioprocess conditions are widely known to heavily influence glycosylation, in particular, β-1,4 galactosylation [13]. Reducing product heterogeneity is key to ensuring the safety and therapeutic efficacy of therapeutic proteins and is, thus, a fundamental aim of biopharmaceutical manufacturing operations.

The invention neutralises the negative effects of varying bioprocess conditions on product β-1,4 galactosylation, thus reducing product heterogeneity:
The nutrient availability issues are addressed by eliminating the consumption of UDP-Gal towards mAb-irrelevant cellular O-linked galactosylation, which accounts for approximately 30% of all UDP-Gal consumed in non-engineered CHO cells [7]. When cellular O-galactosylation is eliminated, more UDP-Gal is made available and product N-galactosylation becomes less dependent on fluctuations in nutrient availability.
The invention reduces the negative impact of metabolite accumulation (e.g. ammonia) or pH shifts/excursions, both of which reduce β4GalT activity, through genetic overexpression of the enzyme. This reduces the sensitivity to changes in the cell culture environment and, in consequence, also reduces product heterogeneity.

2. Increased β-1,4 Galactosylation Enhances the Efficacy of Both Anti-Cancer and Anti-Inflammatory Monoclonal Antibodies (mAbs).

High levels of β-1,4 galactosylated mAb glycans increases antibody-dependent cellular cytotoxicity (ADCC) by up to 30% [14] and complement-dependent cellular cytotoxicity (CDC) by up to 26% [15]. In many commercial products, both cytotoxic mechanisms (ADCC and CDC) define the therapeutic efficacy of anti-cancer mAbs [16, 17].

The invention achieves unprecedented levels of β-1,4 galactosylation on the Fc of mAbs produced by CHO cells—up to 98% β4-galactosylated N-glycans (Table 8 and FIG. 6), thus demonstrating great potential in enhancing the anti-cancer activity of mAb products.

The presence of α-2,6-N-acetylneuraminic acid (α6Neu5Ac) residues on the glycans of antibody therapies have been reported to enhance immune modulation and, thus, increase the efficacy of anti-inflammatory mAbs. Because the addition of α6Neu5Ac requires β-1,4 galactose residues as acceptors, high levels of β-1,4 galactosylation enable increased presence of α6Neu5Ac [19-21]; therefore, the increased levels of product β-1,4 galactosylation achieved by the invention may positively contribute to enhanced efficacy of anti-inflammatory mAbs.

3. Increased β-1,4 Galactosylation Contributes to Enhancing the Serum Half-Life of Therapeutic Proteins.

Therapeutic glycoproteins with higher Neu5Ac content (sialylation) require reduced doses or less frequent dosing because increased sialylation extends the half-life of therapeutic glycoproteins in the patient's serum [22, 23]. Increased sialylation may therefore reduce treatment costs for healthcare providers and patients. Again, β-1,4 galactosylation is intrinsically necessary for the addition of Neu5Ac residues. Therefore, increased levels of β-1,4 galactosylation are required to achieve enhanced serum half-life and, thereby, to improved therapeutic efficacy of therapeutic glycoproteins.

CHO DP12 Cell Line

Referring to FIG. 4 and Tables 2 and 3—CHO DP12 Cell Line. Both cell engineering events are required.

FIG. 4 and Tables 2 and 3 present the distribution of glycans present on the Fc of mAbs produced with six cell lines derived from parental CHO DP12 cells:
(i) CHO DP12 Parental: non-engineered CHO DP12 cells.
(ii) CHO DP12 mCOSMC−: CHO DP12 cells transfected with the CRISPR plasmid in absence of the gRNA sequence targeting COSMC (Mock COSMC− plasmid).
(iii) CHO DP12 COSMC−: CHO DP12 cells transfected with the CRISPR plasmid containing the COSMC-targeting gRNA.
(iv) CHO DP12 mGalT+: CHO DP12 cells transfected with the empty (absence of hβ4GalT1) pUNO plasmid (Mock GalT+ plasmid).
(v) CHO DP12 GalT+: CHO DP12 cells transfected with the hβ4GalT1-containing pUNO plasmid.
(vi) CHO DP12 GalMAX (COSMC−/GalT+): CHO DP12 cells that simultaneously contain the COSMC knockout and hβ4GalT1 expression (transfected/selected for CRISPR knockout of COSMC and transfected/selected for hβ4GalT1 expression).

FIG. 4A presents the mAb Fc glycoform distributions produced by each of the six CHO DP12 cell lines cultured under batch conditions. The data correspond to triplicate cultures for CHO DP12 Parental, duplicate cultures for mCOSMC−, four cultures for COSMC−, five cultures for mGalT+, duplicate cultures for GalT+ and four cultures for CHO DP12 GalMAX. The mAb Fc glycosylation patterns were measured using mass spectrometry (Carillo et al. Journal of Pharmaceutical Analysis. Volume 10, Issue 1, February 2020, Pages 23-34. https://doi.org/10.1016/j.jpha.2019.11.008).

Four major glycoforms are observed:
(i) A2G0F: biantennary, non galactosylated and fucosylated.
(ii) A2G1F: biantennary, mono-galactosylated and fucosylated.
(iii) A2G2F: biantennary, bi-galactosylated and fucosylated.
(iv) A2S1G2F: biantennary, mono-sialylated, bi-galactosylated and fucosylated.

There are no statistically significant differences (two-tailed t-test) among the mAb Fc glycoprofiles produced by the CHO DP12 Parental, CHO DP12 mCOSMC−, CHO DP12 COSMC−, and CHO DP12 mGalT+ cell lines, where the average relative abundances of A2G0F, A2G1F and A2G2F are 47.8%±5%, 43.8%±4.1%, and 8.3%±1.4%, respectively (averages and standard deviations across all replicate cultures for the four cell lines).

The cell lines expressing hβ4GalT1 present a substantial decrease in A2G0F and a concomitant increase in A2G2F. For the CHO DP12 GalT+ cell line, A2G0F is reduced to 7.5%±0.3% and A2G2F increases to 77.9%±1.5%. For the CHO DP12 GalMAX (COSMC−/GalT+) cell line, A2G0F is reduced to 3.8%±0.4% and A2G2F increases to 79.4%±1.8%. Although no statistically significant difference is observed in the bi-galactosylated A2G2F glycoform produced by the GalT+ and GalMAX cell lines, CHO DP12 GalMAX cells produce approximately half of the non-galactosylated A2G0F glycoform generated by GalT+ cells (p<0.01), thus indicating that the knockout of COSMC contributes to increased mAb galactosylation and overall glycan homogeneity.

FIG. 4B presents a comparison between the total non-galactosylated (A2G0F) and galactosylated (sum of A2G1F, A2G2F and A2S1G2F) glycoforms produced by all CHO DP12 cell lines. Again, there is no statistically significant difference in mAb Fc galactosylation among the Parental, COSMC− (Mock), COSMC−, and GalT+(Mock) cell lines, where overall averages (across all four cell lines) for non-galactosylation and galactosylation are 47.8%±5.0% and 52.2%±5.0%, respectively. In contrast, a significant difference in galactosylation is observed between the CHO DP12 GalT+ and GalMAX cell lines, where CHO DP12 GalMAX produces 3.8%±0.4% non-galactosylated and 96.2%±0.6% galactosylated species compared to 7.5%±0.3% and 92.5%±0.3% produced by the CHO DP12 GalT+ cell line. Overall, when deployed in the CHO DP12 cell line, the GalMAX invention delivers a 12-fold decrease in A2G0F and a 9.4-fold increase in A2G2F as well as a 74.1% increase in galactosylated glycoforms, when compared to the parental cell line.

Table 2 provides numerical values for FIG. 4A.

| | Relative abundance ± S.D. (%) | | |
|---|---|---|---|
| | A2G0F | A2G1F | A2G2F |
| DP12 Parental | 44.7 ± 3.1 | 46.8 ± 2.6 | 8.4 ± 0.8 |
| DP12 mCOSMC− | 49.6 ± 4.2 | 43.0 ± 2.2 | 7.1 ± 1.7 |
| DP12 COSMC− | 45.0 ± 6.3 | 45.8 ± 5.5 | 9.1 ± 0.9 |
| DP12 mGalT+ | 51.2 ± 2.4 | 40.6 ± 1.3 | 8.1 ± 1.5 |
| Average | 47.8 ± 5.0 | 43.8 ± 4.1 | 8.3 ± 1.4 |
| DP12 GalT+ | 7.5 ± 0.3 | 12.8 ± 0.2 | 77.9 ± 1.5 |
| DP12 | 3.8 ± 0.4 | 15.5 ± 1.9 | 79.4 ± 1.8 |
| GalMAX | 12-fold reduction | | 9.4-fold increase |

Table 3 provides numerical values for FIG. 4B.

Relative abundance ± S.D. (%)

| | Non-galactosylated | Galactosylated |
|---|---|---|
| DP12 Parental | 44.7 ± 3.1 | 55.2 ± 3.2 |
| DP12 mCOSMC− | 49.6 ± 4.2 | 50.2 ± 4.1 |
| DP12 COSMC− | 45 ± 6.3 | 55.2 ± 6.3 |
| DP12 mGalT+ | 51.2 ± 2.4 | 48.8 ± 2.4 |
| Average | 47.8 ± 5.0 | 52.2 ± 5.0 |
| DP12 GalT+ | 7.5 ± 0.3 | 92.5 ± 0.3 |
| DP12 | 3.8 ± 0.4 | 96.2 ± 0.6 |
| GalMAX | 12-fold reduction | 74.1% increase |

CHO VRC01 Cell Line

Referring to FIG. 5 and Tables 4 and 5—CHO VRC01 Cell Line. Both cell engineering events are required.

FIG. 5 and Tables 4 and 5 present the distribution of glycans present on the Fc of mAbs produced with six CHO VRC01-derived cell lines, which, similarly to their DP12 counterparts, are named VRC01 Parental, VRC01 mCOSMC−, VRC01 COSMC−, VRC01 mGalT+, VRC01 GalT+ and VRC01 GalMAX (COSMC−/GalT+).

FIG. 5A presents the mAb Fc glycoform distributions produced by each of the six CHO VRC01 cell lines cultured under batch conditions. The data correspond to five cultures of VRC01 Parental, a single culture of VRC01 mCOSMC−, four cultures of VRC01 COSMC−, duplicate cultures of VRC01 mGalT+, five cultures of VRC01 GalT+ and triplicate cultures of VRC01 GalMAX. The mAb Fc glycosylation patterns were measured using mass spectrometry method outlined by Carillo et al. (Journal of Pharmaceutical Analysis. Volume 10, Issue 1, February 2020, Pages 23-34. https://doi.org/10.1016/j.jpha.2019.11.008).

The same major glycoforms produced by CHO DP12-derived cell lines are also observed for the CHO VRC01 cell lines (A2G0F, A2G1F, A2G2F, and A2S1G2F). Little or no statistically significant differences (two-tailed t-test) were observed among the mAb Fc glycoprofiles produced by the VRC01 Parental, VRC01 mCOSMC−, VRC01 COSMC−, and VRC01 mGalT+ cell lines, where the average relative abundances of A2G0F, A2G1F and A2G2F are 58.0%±37.4%±4.7%, and 4.6%±1.1%, respectively (averages and standard deviations across all replicate cultures for the four cell lines).

The cell lines expressing hβ4GalT1 present a substantial decrease in A2G0F and a concomitant increase in A2G2F. For the VRC01 GalT+ cell line, A2G0F is reduced to 8.6%±5.0% and A2G2F increases to 72.0%±5.8%. In the VRC01 GalMAX cell line, A2G0F is reduced to 2.4%±0.4% and A2G2F increases to 80.3%±0.6%. The VRC01 GalMAX cell line produces 3.6±2.2-fold less A2G0F than the VRC01 GalT+ cell line, thus indicating that the knockout of COSMC contributes to increased mAb galactosylation and overall glycan homogeneity.

FIG. 5B presents a comparison between the total non-galactosylated (A2G0F) and galactosylated (sum of A2G1F, A2G2F and A2S1G2F) glycoforms produced by all CHO VRC01 cell lines. There are small differences among the mAb Fc glycoprofiles generated by the Parental, mCOSMC−, COSMC−, and mGalT+ cell lines, where overall averages (across all four cell lines) for non-galactosylation and galactosylation are 58.0%±5.7% and 42.0±5.5%, respectively. The cell lines expressing hβ4GalT1 present substantial increases in galactosylation. VRC01 GalT+produces 91.4%±5.0% galactosylated and 8.6%±5.0% non-galactosylated mAb Fc glycans. VRC01 GalMAX generates 97.6%±0.4% galactosylated and only 2.4%±0.4% non-galactosylated mAb Fc glycans. Overall, the GalMAX invention deployed in VRC01 cells delivers a 2.2-fold increase in galactosylation and a 23-fold decrease in non-galactosylated glycans when compared to the parental VRC01 cell line.

Table 4 provides numerical values for FIG. 5A.

| | Relative abundance ± S.D. (%) | | |
|---|---|---|---|
| | A2G0F | A2G1F | A2G2F |
| VRC01 Parental | 55.0 ± 2.6 | 39.8 ± 2.1 | 5.2 ± 0.7 |
| VRC01 mCOSMC− | 59.9 | 35.8 | 4.3 |
| VRC01 COSMC− | 58.3 ± 8.3 | 37.2 ± 5.7 | 4.5 ± 1.1 |
| VRC01 mGalT+ | 63.9 ± 1.9 | 32.6 ± 1.6 | 3.5 ± 0.3 |
| Average | 58.0 ± 5.7 | 37.4 ± 4.7 | 4.6 ± 1.1 |
| VRC01 GalT+ | 8.6 ± 5.0 | 18.5 ± 1.6 | 72.0 ± 5.8 |
| VRC01 GalMAX | 2.4 ± 0.4 | 16.3 ± 1.1 | 80.3 ± 0.6 |
| | 23-fold reduction | | 15.5-fold increase |

Table 5 provides numerical values for FIG. 5B.

Relative abundance ± S.D. (%)

| | Non-galactosylated | Galactosylated |
|---|---|---|
| VRC01 Parental | 55.0 ± 2.6 | 45.0 ± 2.6 |
| VRC01 mCOSMC− | 59.9 | 40.1 |
| VRC01 COSMC− | 58.3 ± 8.3 | 41.7 ± 8.3 |
| VRC01 mGalT+ | 63.9 ± 1.9 | 36.1 ± 1.9 |
| Average | 58.0 ± 5.7 | 42.0 ± 5.5 |
| VRC01 GalT+ | 8.6 ± 5.0 | 91.4 ± 5.0 |
| VRC01 GalMAX | 2.4 ± 0.4 | 97.6 ± 0.4 |
| | 23-fold reduction | 2.2-fold increase |

The results obtained for the CHO DP-12 and VRC01 cell lines are consistent with the mechanisms governing galactosylation. The two key bottlenecks which limit galactosylation are (i) UDP-Gal availability and (ii) β4GalT availability/activity. If there is sufficient UDP-Gal, but β4GalT availability/activity is reduced, then low levels of galactosylation will be observed. Conversely, if β4GalT availability/activity is in excess but there is insufficient UDP-Gal availability, low levels of galactosylation will also be observed. Our overall results indicate that β4GalT availability limits galactosylation in the cell lines where hβ4GalT1 is not expressed (Parental, mCOSMC−, COSMC−, and mGalT+). This bottleneck is alleviated when hβ4GalT1 is ectopically expressed (GalT+ and the GalMAX cell lines), and the increase in overall galactosylation between the GalT+ and GalMAX cell lines is due to the gain in UDP-Gal availability afforded by the COSMC knockout. Therefore, both cell engineering interventions (COSMC knockout and ectopic h(34GalT1 expression) are required to maximise mAb Fc galactosylation and, thus, the GalMAX invention is validated.

Table 6 provides a comparison of Integral of Viable Cells (IVC), specific productivity (qₚ) and product titre for batch cultures of CHO DP12 and CHO VRC01.

| | IVC | qₚ | |
|---|---|---|---|
| | (10⁶ cells | (pg cell⁻¹ | Titre |
| | mL⁻¹ day) | day⁻¹) | (mg L⁻¹) |
| DP12 Parental | 14.3 ± 0.2 | 6.2 ± 0.2 | 123.1 ± 0.1 |
| DP12 COSMC− | 16.1 ± 0.1 | 5.9 ± 0.0 | 128.9 ± 1.7 |
| DP12 GalT+ | 16.5 ± 0.3 | 4.3 ± 0.0 | 104.8 ± 1.2 |
| DP12 GalMAX | 19.5 ± 1.0 | 4.0 ± 0.0 | 133.5 ± 0.2 |
| VRC01 Parental | 48.3 ± 0.9 | 5.0 ± 0.3 | 297.4 ± 3.8 |
| VRC01 COSMC− | 46.5 ± 0.8 | 5.8 ± 0.6 | 314.4 ± 5.2 |
| VRC01 GalT+ | 49.5 ± 0.1 | 5.6 ± 0.4 | 327.5 ± 2.0 |
| VRC01 GalMAX | 47.4 ± 1.1 | 6.4 ± 0.7 | 333.6 ± 5.3 |

Table 7 provides a comparison of Integral of Viable Cells (IVC), specific productivity (qₚ) and product titre for fed-batch cultures of CHO DP12.

| | IVC | qₚ | Titre |
|---|---|---|---|
| | (10⁶ cells mL⁻¹ d) | (pg cell⁻¹ day⁻¹) | (mg L⁻¹) |
| DP12 Parental | 43.6 ± 1.2 | 2.1 ± 0.0 | 155.6 ± 1.5 |
| DP12 COSMC− | 43.0 ± 0.4 | 2.7 ± 0.0 | 182.6 ± 0.6 |
| DP12 GalT+ | 37.4 ± 0.4 | 1.4 ± 0.1 | 109.0 ± 5.0 |
| DP12 GalMAX | 37.7 ± 0.1 | 2.5 ± 0.1 | 157.6 ± 3.8 |

Tables 6 and 7 present a comparison of cell culture KPIs to demonstrate that the GalMAX technology has no negative impact on the productivity of the CHO DP12 and CHO VRC01 cell lines used for the study. Table 6 presents data corresponding to batch cultivation, where DP12 GalMAX presents an increase in integral of viable cells (IVC) from 14.3±0.22 to 19.5±0.95 10⁶ cells mL⁻¹ day when compared with the parental cell line. This increase in IVC results in a reduction in specific productivity (qₚ) from 6.21±0.16 to 4.04±0.02 pg cell⁻¹ day⁻¹, when compared with parental CHO DP12. These changes in IVC and qₚ offset to yield similar titres. DP12 GalMAX achieves a mAb titre of 133.5±0.2 mg L⁻¹, which is slightly higher than the 123.1±0.1 mg L⁻¹ produced by Parental CHO DP12.

The VRC01 GalMAX cell line yield no statistically significant difference in values for IVC when compared with the VRC01 Parental (47.4±1.1 10⁶ cells mL⁻¹ day and 48.3±0.9 10⁶ cells mL⁻¹ day, respectively). Interestingly, VRC01 GalMAX presents an increased qₚ (6.4±0.7 pg cell⁻¹ day⁻¹), when compared with parental CHO DP12 (5.0±0.3 pg cell⁻¹ day⁻¹). The increase in qₚ alongside the similar IVC yields a slight increase in product titre achieved by the VRC01 GalMAX cell line, when compared to the VRC01 parental (333.6±5.3 mg L⁻¹ vs. 297.4±3.8 mg L⁻¹, respectively).

Table 7 compares IVC, qₚ and titre across four CHO DP12-derived cell lines (DP12 Parental, DP12 COSMC−, DP12 GalT+, and DP12 GalMAX) when cultured under fed-batch mode. DP12 GalMAX achieves a slightly lower IVC of 37.7±0.1 10⁶ cells mL⁻¹ day when compared to the 43.6±1.2 10⁶ cells mL⁻¹ day achieved by DP12 Parental. This decreased IVC results in a slightly increased qₚ of 2.5±0.1 pg cell⁻¹ day⁻¹ when compared to the 2.1±0.0 pg cell⁻¹ day⁻¹ of the DP12 parental cell line. These differences in IVC and qₚ offset to yield similar titres of 157.6±3.8 mg L⁻¹ (DP12 GalMAX) and 155.6±1.5 mg L⁻¹ (DP12 Parental).

Overall, Tables 6 and 7 demonstrate that the GalMAX technology has no negative impact on cell culture KPIs—in all cases, the GalMAX technology yielded titres that are higher than the parental cell lines.

Comparison of GalMAX with Other Technologies

Referring to FIG. 6 and Table 8. GalMAX performs comparably or outperforms other available technologies.

FIG. 6 and Table 8 compare the GalMAX invention with other technologies that have been developed to maximise mAb Fc galactosylation. UMG feeding refers to uridine-manganese-galactose cell culture supplementation [1, 2], β4GalT expression refers to ectopic expression of the β4GalT enzyme [19, 24, 25], and Enzymatic remodelling refers to in vitro enzymatic modification of mAb Fc glycans [4, 14].

The GalMAX invention delivers between 96.2% and 97.6% overall galactosylation (DP12 and VRC01 cell lines, respectively). These values are comparable with the 98% total galactosylation achieved by the cell engineering strategy of Schulz et al. [25] and the in vitro methods of Thomann et al. [14] and Tayi & Butler [4]. GalMAX considerably outperforms the cell engineering strategies by Raymond et al. and Chang et al. [24], which yield values of 73% and 87%, respectively. GalMAX also outperforms UMG feeding strategies, which report between 48% and 67% total mAb Fc galactosylation [1, 2].

The GalMAX invention yields up to 80% bi-galactosylated mAb Fc glycans, which is comparable with the value of 83% obtained by Thomann et al. [14] (in vitro). The levels of mAb Fc bi-galactosylation obtained by Schulz et al. [25] (cell engineering) and Tayi & Butler (in vitro) are slightly higher than those achieved by the GalMAX invention. GalMAX produces a considerably larger fraction of bi-galactosylated mAb Fc glycans than those reported for UMG feeding [1, 2], and other cell engineering strategies [19, 24].

Table 8 compares the performance of GalMAX with existing technologies.

| | Total galactosylation | | | Total bi-galactosylation | | | | |
|---|---|---|---|---|---|---|---|---|
| | | After | Fold | | After | Fold | Titre | |
| | Initial | Treatment | Increase | Initial | Treatment | Increase | (g/L) | Refs. |
| UMG feeding | 9.4% | 48.1% | 5.1 | 0.5% | 9.7% | 19.4 | 3.20 | [1] |
| | 35.6% | 67.1% | 1.9 | 4.5% | 19.1% | 4.2 | 0.32 | [2] |
| β4GalT | 37.4% | 72.9% | 2.0 | 5.1% | 48.8% | 9.6 | 0.02 | [19] |
| expression | 10.2% | 86.7% | 8.5 | 1.4% | 62.3% | 44.5 | 0.05 | [24] |
| | 45.1% | 97.9% | 2.2 | 6.8% | 87.2% | 12.8 | 0.30 | [25] |
| Enzymatic | 40.3% | 98.2% | 2.4 | 4.4% | 82.8% | 18.8 | N/A | [14] |
| remodelling | 39.0% | 98.0% | 2.5 | 5.0% | 92.5% | 18.5 | N/A | [4] |
| DP12 GalMAX | 55.2% | 96.2% | 1.7 | 8.4% | 79.4% | 9.4 | 0.13 | |
| VRC01 GalMAX | 45.0% | 97.6% | 2.2 | 5.2% | 80.3% | 15.5 | 0.33 | |

Fed-Batch Culture of CHO DP12

Referring to FIG. 7 and Table 9. GalMAX CHO DP12 Cell Line. Both cell engineering events are required and the level of mono- and bi-galactosylation is maintained across batch and fed-batch culture.

FIG. 7 and Table 9 present the distribution of glycans present on the Fc of mAbs produced with the four engineered cell lines derived from CHO DP12 (DP12 Parental, DP12 COSMC−, DP12 GalT+ and DP12 GalMAX) cultured under fed-batch mode. The data correspond to duplicate cultures of each cell line. The mAb Fc glycosylation patterns were measured using mass spectrometry method outlined by Carillo et al. (Journal of Pharmaceutical Analysis. Volume 10, Issue 1, February 2020, Pages 23-34. https//doi.org/10.1016/j.jpha.2019.11.008).

FIG. 7A shows that the same major glycoforms observed for batch culture are also observed for fed-batch mode (A2G0F, A2G1F, A2G2F, and A2S1G2F). The DP12 COSMC− cell line produces marginally lower non-galactosylated glycoform A2G0F and higher mono-galactosylated A2G1F glycans. No statistically significant differences are observed in the fraction of bi-galactosylated A2G2F glycan. These results indicate that COSMC knockout contribute to enhanced product glycosylation in cells cultured under fed-batch operation.

FIG. 7A shows that, as with batch cultivation, CHO DP12 expressing hβ4GalT1 present a substantial decrease in A2G0F (from 51.4±1.2 to 11.8±2.0) and a concomitant increase in A2G2F (from 7.2±0.3 to 57.5±8.6). In the CHO DP12 GalMAX cell line, A2G0F is reduced to 1.7%±0.2% and A2G2F increases to 80.0%±0.6%. The DP12 GalMAX cell line produces 7.1±1.5-fold less A2G0F than the DP12 GalT+ cell line, thus indicating that the knockout of COSMC substantially contributes to increased mAb galactosylation and overall glycan homogeneity.

FIG. 7B presents a comparison between the total non galactosylated (Man5+A2G0F) and galactosylated (sum of A2G1F, A2G2F and A2S1G2F) glycoforms produced by the four DP12-derived cell lines when cultured in fed-batch mode. Minor differences are observed between the galactosylation of product generated by DP12 Parental and DP12 COSMC− cells (47.7%±1.2% and 50.7%±1.4%, respectively). DP12 GalT+produces 77.1%±11.0% galactosylated and 22.6%±8.1% non-galactosylated mAb Fc glycans. DP12 GalMAX generates 91.8%±0.3% galactosylated and 8.2%±0.2% mAb Fc glycans. Overall, the GalMAX invention deployed in CHO DP12 cells cultured under fed-batch mode yield a 1.9-fold increase in galactosylation and a 6.4-fold decrease in non-galactosylated glycans when compared to the parental DP12 cell line.

Table 9 provides numerical values for FIG. 7A.

| | Relative abundance ± S.D. (%) | | |
|---|---|---|---|
| | A2G0F | A2G1F | A2G2F |
| DP12 Parental | 51.4 ± 1.2 | 40.5 ± 0.9 | 7.2 ± 0.3 |
| DP12 COSMC− | 48.0 ± 0.9 | 43.2 ± 0.6 | 7.5 ± 0.8 |
| DP12 GalT+ | 11.8 ± 2.0 | 18.9 ± 1.4 | 57.5 ± 8.6 |
| DP12 GalMAX | 1.7 ± 0.2 | 11.1 ± 0.2 | 80.0 ± 0.6 |
| | 12-fold | 3.6-fold | 11-fold |
| | reduction | reduction | increase |

### DISCUSSION

The key aspect that sets the proposed invention aside from other cell glycoengineering strategies is the combination of knocking out COSMC expression and ectopically expressing β4GalT1.

Knocking out COSMC provides additional UDP-Gal co-substrate required for product β-1,4 galactosylation without additional manipulations or feeding strategies that are known to negatively impact cell growth and productivity. Computational studies from our group indicate that cellular O-linked galactosylation is the largest sink for UDP-Gal consumption in CHO cells—over 30% of all UDP-Gal is consumed goes towards cellular O-linked galactosylation [7].

Simultaneously overexpressing the β4GalT1 enzyme provides additional cellular machinery to catalyse the reaction whereby β-1,4 galactose is added to product N-linked glycans. The baseline levels of endogenous β4GalTs in CHO cells are insufficient to perform extensive product β-1,4 galactosylation. Alongside this, commonly observed variations in cell culture conditions (e g ammonia accumulation or pH shifts) may negatively impact the activity of endogenous β4GalTs. Therefore, ectopically expressing β4GalT1 provides additional machinery to achieve higher product β-1,4 galactosylation and simultaneously limits the negative impacts varying cell culture conditions have on enzymatic activity.

β-1,4 galactosylation has only been recently identified as a key determining factor of the therapeutic efficacy of mAb products. Thus, there are great opportunities to enhance the pharmacokinetics and pharmacodynamics of these products by maximising this glycan motif. This invention presents an entirely novel and facile genetic engineering strategy that maximises the β-1,4 galactosylation of mAbs. In the context of biopharmaceutical manufacturing, the invention has broad scope of implementation and could rapidly contribute to enhancing the safety and efficacy of life-saving medicines that are the highest-grossing class of pharmaceutical products.

Sequences
β1,4-galactosyltransferase I isoform 1
amino acid sequence
>NP_001488.2 beta-1,4-galactosyltransferase 1
isoform 1 [Homo sapiens]
(SEQ ID NO: 1)
MRLREPLLSGSAAMPGASLQRACRLLVAVCALHLGVTLVY
YLAGRDLSRLPQLVGVSTPLQGGSNSAAAIGQSSGELRTG
GARPPPPLGASSQPRPGGDSSPVVDSGPGPASNLTSVPVP
HTTALSLPACPEESPLLVGPMLIEFNMPVDLELVAKQNPN
VKMGGRYAPRDCVSPHKVAIIIPFRNRQEHLKYWLYYLHP
VLQRQQLDYGIYVINQAGDTIFNRAKLLNVGFQEALKDYD
YTCFVFSDVDLIPMNDHNAYRCFSQPRHISVAMDKFGFSL
PYVQYFGGVSALSKQQFLTINGFPNNYWGWGGEDDDIFNR
LVFRGMSISRPNAVVGRCRMIRHSRDKKNEPNPQRFDRIA
HTKETMLSDGLNSLTYQVLDVQRYPLYTQITVDIGTPS
β1,4-galactosyltransferase I DNA/encoding
sequence
>NM_001497.4 Homo sapiens
beta-1,4-galactosyltransferase 1 (B4GALT1),
transcript variant 1, mRNA
(SEQ ID NO: 2)
GCTCCCAGGTCTGGCTGGCTGGAGGAGTCTCAGCTCTCAG
CCGCTCGCCCGCCCCCGCTCCGGGCCCTCCCCTAGTCGCC
GCTGTGGGGCAGCGCCTGGCGGGCGGCCCGCGGGCGGGTC
GCCTCCCCTCCTGTAGCCCACACCCTTCTTAAAGCGGCGG
CGGGAAGATGAGGCTTCGGGAGCCGCTCCTGAGCGGCAGC
GCCGCGATGCCAGGCGCGTCCCTACAGCGGGCCTGCCGCC
TGCTCGTGGCCGTCTGCGCTCTGCACCTTGGCGTCACCCT
CGTTTACTACCTGGCTGGCCGCGACCTGAGCCGCCTGCCC
CAACTGGTCGGAGTCTCCACACCGCTGCAGGGCGGCTCGA
ACAGTGCCGCCGCCATCGGGCAGTCCTCCGGGGAGCTCCG
GACCGGAGGGGCCCGGCCGCCGCCTCCTCTAGGCGCCTCC
TCCCAGCCGCGCCCGGGTGGCGACTCCAGCCCAGTCGTGG
ATTCTGGCCCTGGCCCCGCTAGCAACTTGACCTCGGTCCC
AGTGCCCCACACCACCGCACTGTCGCTGCCCGCCTGCCCT
GAGGAGTCCCCGCTGCTTGTGGGCCCCATGCTGATTGAGT
TTAACATGCCTGTGGACCTGGAGCTCGTGGCAAAGCAGAA
CCCAAATGTGAAGATGGGCGGCCGCTATGCCCCCAGGGAC
TGCGTCTCTCCTCACAAGGTGGCCATCATCATTCCATTCC
GCAACCGGCAGGAGCACCTCAAGTACTGGCTATATTATTT
GCACCCAGTCCTGCAGCGCCAGCAGCTGGACTATGGCATC
TATGTTATCAACCAGGCGGGAGACACTATATTCAATCGTG
CTAAGCTCCTCAATGTTGGCTTTCAAGAAGCCTTGAAGGA
CTATGACTACACCTGCTTTGTGTTTAGTGACGTGGACCTC
ATTCCAATGAATGACCATAATGCGTACAGGTGTTTTTCAC
AGCCACGGCACATTTCCGTTGCAATGGATAAGTTTGGATT
CAGCCTACCTTATGTTCAGTATTTTGGAGGTGTCTCTGCT
CTAAGTAAACAACAGTTTCTAACCATCAATGGATTTCCTA
ATAATTATTGGGGCTGGGGAGGAGAAGATGATGACATTTT
TAACAGATTAGTTTTTAGAGGCATGTCTATATCTCGCCCA
AATGCTGTGGTCGGGAGGTGTCGCATGATCCGCCACTCAA
GAGACAAGAAAAATGAACCCAATCCTCAGAGGTTTGACCG
AATTGCACACACAAAGGAGACAATGCTCTCTGATGGTTTG
AACTCACTCACCTACCAGGTGCTGGATGTACAGAGATACC
CATTGTATACCCAAATCACAGTGGACATCGGGACACCGAG
CTAGCGTTTTGGTACACGGATAAGAGACCTGAAATTAGCC
AGGGACCTCTGCTGTGTGTCTCTGCCAATCTGCTGGGCTG
GTCCCTCTCATTTTTACCAGTCTGAGTGACAGGTCCCCTT
CGCTCATCATTCAGATGGCTTTCCAGATGACCAGGACGAG
TGGGATATTTTGCCCCCAACTTGGCTCGGCATGTGAATTC
TTAGCTCTGCAAGGTGTTTATGCCTTTGCGGGTTTCTTGA
TGTGTTCGCAGTGTCACCCCAGAGTCAGAACTGTACACAT
CCCAAAATTTGGTGGCCGTGGAACACATTCCCGGTGATAG
AATTGCTAAATTGTCGTGAAATAGGTTAGAATTTTTCTTT
AAATTATGGTTTTCTTATTCGTGAAAATTCGGAGAGTGCT
GCTAAAATTGGATTGGTGTGATCTTTTTGGTAGTTGTAAT
TTAACAGAAAAACACAAAATTTCAACCATTCTTAATGTTA
CGTCCTCCCCCCACCCCCTTCTTTCAGTGGTATGCAACCA
CTGCAATCACTGTGCATATGTCTTTTCTTAGCAAAAGGAT
TTTAAAACTTGAGCCCTGGACCTTTTGTCCTATGTGTGTG
GATTCCAGGGCAACTCTAGCATCAGAGCAAAAGCCTTGGG
TTTCTCGCATTCAGTGGCCTATCTCCAGATTGTCTGATTT
CTGAATGTAAAGTTGTTGTGTTTTTTTTTAAATAGTAGTT
TGTAGTATTTTAAAGAAAGAACAGATCGAGTTCTAATTAT
GATCTAGCTTGATTTTGTGTTGATCCAAATTTGCATAGCT
GTTTAATGTTAAGTCATGACAATTTATTTTTCTTGGCATG
CTATGTAAACTTGAATTTCCTATGTATTTTTATTGTGGTG
TTTTAAATATGGGGAGGGGTATTGAGCATTTTTTAGGGAG
AAAAATAAATATATGCTGTAGTGGCCACAAATAGGCCTAT
GATTTAGCTGGCAGGCCAGGTTTTCTCAAGAGCAAAATCA
CCCTCTGGCCCCTTGGCAGGTAAGGCCTCCCGGTCAGCAT
TATCCTGCCAGACCTCGGGGAGGATACCTGGGAGACAGAA
GCCTCTGCACCTACTGTGCAGAACTCTCCACTTCCCCAAC
CCTCCCCAGGTGGGCAGGGCGGAGGGAGCCTCAGCCTCCT
TAGACTGACCCCTCAGGCCCCTAGGCTGGGGGGTTGTAAA
TAACAGCAGTCAGGTTGTTTACCAGCCCTTTGCACCTCCC
CAGGCAGAGGGAGCCTCTGTTCTGGTGGGGGCCACCTCCC
TCAGAGGCTCTGCTAGCCACACTCCGTGGCCCACCCTTTG
TTACCAGTTCTTCCTCCTTCCTCTTTTCCCCTGCCTTTCT
CATTCCTTCCTTCGTCTCCCTTTTTGTTCCTTTGCCTCTT
GCCTGTCCCCTAAAACTTGACTGTGGCACTCAGGGTCAAA
CAGACTATCCATTCCCCAGCATGAATGTGCCTTTTAATTA
GTGATCTAGAAAGAAGTTCAGCCGAACCCACACCCCAACT
CCCTCCCAAGAACTTCGGTGCCTAAAGCCTCCTGTTCCAC
CTCAGGTTTTCACAGGTGCTCCCACCCCAGTTGAGGCTCC
CACCCACAGGGCTGTCTGTCACAAACCCACCTCTGTTGGG
AGCTATTGAGCCACCTGGGATGAGATGACACAAGGCACTC
CTACCACTGAGCGCCTTTGCCAGGTCCAGCCTGGGCTCAG
GTTCCAAGACTCAGCTGCCTAATCCCAGGGTTGAGCCTTG
TGCTCGTGGCGGACCCCAAACCACTGCCCTCCTGGGTACC
AGCCCTCAGTGTGGAGGCTGAGCTGGTGCCTGGCCCCAGT
CTTATCTGTGCCTTTACTGCTTTGCGCATCTCAGATGCTA
ACTTGGTTCTTTTTCCAGAAGCCTTTGTATTGGTTAAAAA
TTATTTTCCATTGCAGAAGCAGCTGGACTATGCAAAAAGT
ATTTCTCTGTCAGTTCCCCACTCTATACCAAGGATATTAT
TAAAACTAGAAATGACTGCATTGAGAGGGAGTTGTGGGAA
ATAAGAAGAATGAAAGCCTCTCTTTCTGTCCGCAGATCCT
GACTTTTCCAAAGTGCCTTAAAAGAAATCAGACAAATGCC
CTGAGTGGTAACTTCTGTGTTATTTTACTCTTAAAACCAA
ACTCTACCTTTTCTTGTTGTTTTTTTTTTTTTTTTTTTTT
TTTTTTTGGTTACCTTCTCATTCATGTCAAGTATGTGGTT
CATTCTTAGAACCAAGGGAAATACTGCTCCCCCCATTTGC
TGACGTAGTGCTCTCATGGGCTCACCTGGGCCCAAGGCAC
AGCCAGGGCACAGTTAGGCCTGGATGTTTGCCTGGTCCGT
GAGATGCCGCGGGTCCTGTTTCCTTACTGGGGATTTCAGG
GCTGGGGGTTCAGGGAGCATTTCCTTTTCCTGGGAGTTAT
GACCGCGAAGTTGTCATGTGCCGTGCCCTTTTCTGTTTCT
GTGTATCCTATTGCTGGTGACTCTGTGTGAACTGGCCTTT
GGGAAAGATCAGAGAGGGCAGAGGTGGCACAGGACAGTAA
AGGAGATGCTGTGCTGGCCTTCAGCCTGGACAGGGTCTCT
GCTGACTGCCAGGGGCGGGGGCTCTGCATAGCCAGGATGA
CGGCTTTCATGTCCCAGAGACCTGTTGTGCTGTGTATTTT
GATTTCCTGTGTATGCAAATGTGTGTATTTACCATTGTGT
AGGGGGCTGTGTCTGATCTTGGTGTTCAAAACAGAACTGT
ATTTTTGCCTTTAAAATTAAATAATATAACGTGAATAAAT
GACCCTATCTTTGTAA

### REFERENCES

All references herein are incorporated by reference.
1. Gramer, M. J., et al., Modulation of antibody galactosylation through feeding of uridine, manganese chloride, and galactose. Biotechnol Bioeng, 2011. 108(7): p. 1591-602.
2. Grainger, R. K. and D. C. James, CHO cell line specific prediction and control of recombinant monoclonal antibody N-glycosylation. Biotechnol Bioeng, 2013. 110(11): p. 2970-83.
3. Dekkers, G., et al., Multi-level glyco-engineering techniques to generate IgG with defined Fc-glycans. Sci Rep, 2016. 6: p. 36964.
4. Tayi, V. S. and M. Butler, Solid-Phase Enzymatic Remodeling Produces High Yields of Single Glycoform Antibodies. Biotechnol J, 2018. 13(4): p. e1700381.
5. Yang, Z., et al., The GalNAc-type O-Glycoproteome of CHO cells characterized by the SimpleCell strategy. Mol Cell Proteomics, 2014. 13(12): p. 3224-35.
6. Ronda, C., et al., Accelerating genome editing in CHO cells using CRISPR Cas9 and CRISPy, a web-based target finding tool. Biotechnol Bioeng, 2014. 111(8): p. 1604-16.
7. del Val, I. J., K. M. Polizzi, and C. Kontoravdi, A theoretical estimate for nucleotide sugar demand towards Chinese Hamster Ovary cellular glycosylation. Sci Rep, 2016. 6: p. 28547.
8. Ran, F. A., et al., Genome engineering using the CRISPR-Cas9 system. Nat Protoc, 2013. 8(11): p. 2281-2308.
9. Farrell, A., et al., Monoclonal antibody sequence assessment using a hybrid quadrupole-Orbitrap mass spectrometer. Analytical Methods, 2018. 10(25): p. 3100-3109.
10. Raju, T. S. and R. E. Jordan, Galactosylation variations in marketed therapeutic antibodies. MAbs, 2012. 4(3): p. 385-91.
11. Planinc, A., et al., Batch-to-batch N-glycosylation study of infliximab, trastuzumab and bevacizumab, and stability study of bevacizumab. Eur J Hosp Pharm, 2017. 24(5): p. 286-292.
12. Fournier, J., A Review of Glycan Analysis Requirements. Biopharm International, 2015. 28(10): p. 32-37.
13. Bossier, P., S. F. Khattak, and Z. J. Li, Optimal and consistent protein glycosylation in mammalian cell culture. Glycobiology, 2009. 19(9): p. 936-49.
14. Thomann, M., et al., Fc-galactosylation modulates antibody-dependent cellular cytotoxicity of therapeutic antibodies. Mol Immunol, 2016. 73: p. 69-75.
15. Peschke, B., et al., Fc-Galactosylation of Human Immunoglobulin Gamma Isotypes Improves C1 q Binding and Enhances Complement-Dependent Cytotoxicity. Front Immunol, 2017. 8: p. 646.
16. Natsume, A., R. Niwa, and M. Satoh, Improving effector functions of antibodies for cancer treatment: Enhancing ADCC and CDC. Drug Des Devel Ther, 2009. 3: p. 7-16.
17. Rogers, L. M., S. Veeramani, and G. J. Weiner, Complement in monoclonal antibody therapy of cancer. Immunol Res, 2014. 59(1-3): p. 203-10.
18. Anthony, R. M., et al., Recapitulation of IVIG anti-inflammatory activity with a recombinant IgG Fc. Science, 2008. 320(5874): p. 373-6.
19. Raymond, C., et al., Production of alpha2,6-sialylated IgG1 in CHO cells. MAbs, 2015. 7(3): p. 571-83.
20. Weikert, S., et al., Engineering Chinese hamster ovary cells to maximize sialic acid content of recombinant glycoproteins. Nat Biotechnol, 1999. 17(11): p. 1116-21.
21. Fukuta, K., et al., The widespread effect of beta 1,4-galactosyltransferase on N-glycan processing. Arch Biochem Biophys, 2001. 392(1): p. 79-86.
22. Kontermann, R. E., Half-life extended biotherapeutics. Expert Opin Biol Ther, 2016. 16(7): p. 903-15.
23. Bas, M., et al., Fc Sialylation Prolongs Serum Half-Life of Therapeutic Antibodies. J Immunol, 2019. 202(5): p. 1582-1594.
24. Chang, M. M., et al., Small-molecule control of antibody N-glycosylation in engineered mammalian cells. Nat Chem Biol, 2019. 15(7): p. 730-736.
25. Schulz, M. A., et al., Glycoengineering design options for IgG1 in CHO cells using precise gene editing. Glycobiology, 2018. 28(7): p. 542-549.

1. A cell, wherein the cell is modified to:
reduce O-GalNAc galactosylation activity in the cell by reduction of functional COSMC molecular chaperone in the cell and/or by reduction of functional T-synthase in the cell; and
overexpress β1,4-galactosyltransferase in the cell.

2. The cell according to claim 1, wherein the modification comprises a knockout of the COSMC molecular chaperone or wherein translation from the COSMC molecular chaperone gene is supressed.

3. The cell according to claim 1, wherein the modification comprises a knockout of functional T-synthase in the cell or wherein translation from the T-synthase gene is supressed.

4. The cell according to claim 1, wherein the cell is transformed with nucleic acid encoding β1,4-galactosyltransferase.

5. The cell according to claim 1, wherein the β1,4-galactosyltransferase comprises human β4GalT1.

6. The cell according to claim 1, wherein the cell is selected from CHO, HEK, NS0, SP2/0, PER.C6, Sf9, VERY, BH, HeLa, COS, MDCK, 293, 293T, 3T3, WI38, BT483, Hs578T, HTB2, BT20, T47D, CRL7030 and Hs578Bst.

7. The cell according to claim 1, wherein the cell is modified to express a polypeptide product, wherein the polypeptide product is a heterologous polypeptide.

8. (canceled)

9. The cell according to claim 1, wherein the cell is modified to express a polypeptide product, wherein the polypeptide product comprises one or more, or all the peptides of a therapeutic selected from the group comprising [fam-]trastuzumab deruxtecan, Leronlimab, Narsoplimab, REGNEB3, Sacituzumab govitecan, Tafasitamab, Inebilizumab, Satralizumab, Eptinezumab, Isatuximab, Teprotumumab, Crizanlizumab, Enfortumab vedotin, Polatuzumab vedotin, Risankizumab, Romosozumab, Burosumab, Cemiplimab, Emapalumab, emapalumab-lzsg, Erenumab, Fremanezumab, Galcanezumab, Gemtuzumab ozogamicin, Ibalizumab, ibalizumab-uiyk, Lanadelumab, Mogamulizumab, Ravulizumab (ALXN1210), Tildrakizumab, Avelumab, Benralizumab, Brodalumab, Dupilumab, Durvalumab, Emicizumab, Guselkumab, lnotuzumab ozogamicin, Ocrelizumab, Sarilumab, Atezolizumab, Bezlotoxumab, Ixekizumab, Obiltoxaximab, Olaratumab, Reslizumab, Alirocumab, Daratumumab, Dinutuximab, Elotuzumab, Evolocumab, Mepolizumab, Necitumumab, Secukinumab, Nivolumab, Pembrolizumab, Ramucirumab, Siltuximab, Vedolizumab, Alemtuzumab, Obinutuzumab, Ado-trastuzumab emtansine, Pertuzumab, Raxibacumab, Belimumab, Brentuximab vedotin, Ipilimumab, Denosumab, Canakinumab, Golimumab, Ofatumumab, Tocilizumab, Ustekinumab, Eculizumab, Panitumumab, Bevacizumab, Cetuximab, Natalizumab, Omalizumab, Adalimumab, Ibritumomab tiuxetan, Basiliximab, Infliximab, Palivizumab, Trastuzumab, Rituximab, and Abciximab.

10. The cell according to claim 1, wherein the cell is modified to express a polypeptide product, wherein the polypeptide product comprises a component of a viral vector such as rAAV.

11. A method of modifying a cell, wherein the cell is modified to enhance β-1,4 galactosylation of a polypeptide product, the modifications comprising:
reducing O-GalNAc galactosylation activity in the cell by reduction of functional COSMC molecular chaperone in the cell and/or by reduction of functional T-synthase in the cell; and
modification of the cell to provide overexpression of β1,4-galactosyltransferase in the cell.

12. The method according to claim 11, wherein the method comprises the step of transforming the cell with nucleic acid encoding the β1,4-galactosyltransferase.

13. The method according to claim 11, wherein the method comprises modifying the gene(s) encoding the COSMC molecular chaperone and/or T-synthase, or modifying regulatory elements thereof in order to knockout the expression thereof.

14. The method according to claim 11, wherein the method further comprises modifying the cell to express a polypeptide product.

15. A method of producing a polypeptide product, the method comprising providing a cell according to claim 1, or obtaining a modified cell according to claim 11, and culturing the cell for expression of the polypeptide product.

16. The method according to claim 15, wherein the polypeptide product is produced to have at least 80% β-1,4 galactosylation and/or at least 50% bi-galactosylated; or
wherein the polypeptide product is produced to have at least a 0.5 fold increased β-1,4 galactosylation and/or at least 0.5 fold increased bi-galactosylation.

17. The method according to any of claim 15 or 16, wherein the cells are cultured with a UMG feeding strategy comprising or consisting of the addition of uridine, manganese and galactose in the cell culture media.

18-20. (canceled)

21. A plasmid encoding:
a genetic modification element(s) that is targeted to knock-out or reduce functional COSMC molecular chaperone and/or T-synthase expression in a cell;
b4GalT for expression in the cell; and
optionally a selection marker, such as an antibiotic resistance gene.

22. A kit comprising:
a first plasmid encoding:
a genetic modification element(s) that is targeted to knock-out or reduce functional COSMC molecular chaperone and/or T-synthase expression in a cell; and
a second plasmid encoding b4GalT for expression in a cell and optionally a selection marker, such as an antibiotic resistance gene.

23. The kit according to claim 22, further comprising a plasmid encoding a polypeptide product for expression.

24. The kit according to claim 22, wherein the genetic modification element(s) comprise zinc finger nuclease, TALEN (transcription activator-like effector nuclease), guide RNA (gRNA) and Cas9 (for CRISPR modification), silencing RNAs, or homologous recombination cassettes with antibiotic resistance genes.


SEQUENCE LISTING (2 sequences)

SEQ ID NO: 1 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 2 - DNA, 4176 bp, Homo sapiens
gctcccaggtctggctggctggaggagtctcagctctcagccgctcgcccgcccccgctccgggccctcccctagtcgccgctgtggggcagcgcctggcgggcggcccgcgggcgggtcgcctcccctcctgtagcccacacccttcttaaagcggcggcgggaagatgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagcgttttggtacacggataagagacctgaaattagccagggacctctgctgtgtgtctctgccaatctgctgggctggtccctctcatttttaccagtctgagtgacaggtccccttcgctcatcattcagatggctttccagatgaccaggacgagtgggatattttgcccccaacttggctcggcatgtgaattcttagctctgcaaggtgtttatgcctttgcgggtttcttgatgtgttcgcagtgtcaccccagagtcagaactgtacacatcccaaaatttggtggccgtggaacacattcccggtgatagaattgctaaattgtcgtgaaataggttagaatttttctttaaattatggttttcttattcgtgaaaattcggagagtgctgctaaaattggattggtgtgatctttttggtagttgtaatttaacagaaaaacacaaaatttcaaccattcttaatgttacgtcctccccccacccccttctttcagtggtatgcaaccactgcaatcactgtgcatatgtcttttcttagcaaaaggattttaaaacttgagccctggaccttttgtcctatgtgtgtggattccagggcaactctagcatcagagcaaaagccttgggtttctcgcattcagtggcctatctccagattgtctgatttctgaatgtaaagttgttgtgtttttttttaaatagtagtttgtagtattttaaagaaagaacagatcgagttctaattatgatctagcttgattttgtgttgatccaaatttgcatagctgtttaatgttaagtcatgacaatttatttttcttggcatgctatgtaaacttgaatttcctatgtatttttattgtggtgttttaaatatggggaggggtattgagcattttttagggagaaaaataaatatatgctgtagtggccacaaataggcctatgatttagctggcaggccaggttttctcaagagcaaaatcaccctctggccccttggcaggtaaggcctcccggtcagcattatcctgccagacctcggggaggatacctgggagacagaagcctctgcacctactgtgcagaactctccacttccccaaccctccccaggtgggcagggcggagggagcctcagcctccttagactgacccctcaggcccctaggctggggggttgtaaataacagcagtcaggttgtttaccagccctttgcacctccccaggcagagggagcctctgttctggtgggggccacctccctcagaggctctgctagccacactccgtggcccaccctttgttaccagttcttcctccttcctcttttcccctgcctttctcattccttccttcgtctccctttttgttcctttgcctcttgcctgtcccctaaaacttgactgtggcactcagggtcaaacagactatccattccccagcatgaatgtgccttttaattagtgatctagaaagaagttcagccgaacccacaccccaactccctcccaagaacttcggtgcctaaagcctcctgttccacctcaggttttcacaggtgctcccaccccagttgaggctcccacccacagggctgtctgtcacaaacccacctctgttgggagctattgagccacctgggatgagatgacacaaggcactcctaccactgagcgcctttgccaggtccagcctgggctcaggttccaagactcagctgcctaatcccagggttgagccttgtgctcgtggcggaccccaaaccactgccctcctgggtaccagccctcagtgtggaggctgagctggtgcctggccccagtcttatctgtgcctttactgctttgcgcatctcagatgctaacttggttctttttccagaagcctttgtattggttaaaaattattttccattgcagaagcagctggactatgcaaaaagtatttctctgtcagttccccactctataccaaggatattattaaaactagaaatgactgcattgagagggagttgtgggaaataagaagaatgaaagcctctctttctgtccgcagatcctgacttttccaaagtgccttaaaagaaatcagacaaatgccctgagtggtaacttctgtgttattttactcttaaaaccaaactctaccttttcttgttgttttttttttttttttttttttttttttggttaccttctcattcatgtcaagtatgtggttcattcttagaaccaagggaaatactgctccccccatttgctgacgtagtgctctcatgggctcacctgggcccaaggcacagccagggcacagttaggcctggatgtttgcctggtccgtgagatgccgcgggtcctgtttccttactggggatttcagggctgggggttcagggagcatttccttttcctgggagttatgaccgcgaagttgtcatgtgccgtgcccttttctgtttctgtgtatcctattgctggtgactctgtgtgaactggcctttgggaaagatcagagagggcagaggtggcacaggacagtaaaggagatgctgtgctggccttcagcctggacagggtctctgctgactgccaggggcgggggctctgcatagccaggatgacggctttcatgtcccagagacctgttgtgctgtgtattttgatttcctgtgtatgcaaatgtgtgtatttaccattgtgtagggggctgtgtctgatcttggtgttcaaaacagaactgtatttttgcctttaaaattaaataatataacgtgaataaatgaccctatctttgtaa